(19) United States
(12) Patent Application Publication
(10) Pub. No.: US 2024/0071598 A1
(43) Pub. Date: Feb. 29, 2024

(54) METHODS AND SYSTEMS FOR ORDERED FOOD PREFERENCES ACCOMPANYING SYMPTOMATIC INPUTS

(71) Applicant: KPN INNOVATIONS, LLC., Lakewood, CO (US)

(72) Inventor: Kenneth Neumann, Lakewood, CO (US)

(73) Assignee: KPN INNOVATIONS, LLC., Lakewood, CO (US)

(21) Appl. No.: 18/387,266

(22) Filed: Nov. 6, 2023

Related U.S. Application Data
(63) Continuation-in-part of application No. 17/243,670, filed on Apr. 29, 2021, now Pat. No. 11,837,351.
Application No. 17/243,670 is a continuation of application No. 16/887,319, filed on May 29, 2020, now Pat. No. 11,075,001.

(51) Int. Cl. G16H 20/60; G06F 16/2457; G06N 20/00; G06Q 10/0875; G06Q 30/0601; G16H 10/60; G16H 50/20; G16H 70/60

(52) U.S. Cl. CPC G16H 20/60; G06F 16/24578; G06N 20/00; G06Q 10/0875; G06Q 30/0633; G16H 10/60; G16H 50/20; G16H 70/60

(57) ABSTRACT

A system for ordered food preferences accompanying symptomatic inputs, the system including a computing device, the computing device designed and configured to retrieve a food profile pertaining to a user; select a first food element as a function of the food profile; select a second food element as a function of the first food element; create a food preference menu wherein the food preference menu contains the first food element and the second food element; and modify the food preference menu as a function of an entry contained within a symptomatic database.

### CROSS-REFERENCE TO RELATED APPLICATIONS

This continuation-in-part application claims the benefit of priority of U.S. Non-Provisional patent application Ser. No. 17/243,670 filed on Apr. 29, 2021 and entitled “METHODS AND SYSTEMS FOR ORDERED FOOD PREFERENCES ACCOMPANYING SYMPTOMATIC INPUTS,” which is a continuation application claims the benefit of priority of U.S. Non-Provisional patent application Ser. No. 16/887,319 filed on May 29, 2020 and entitled “METHODS AND SYSTEMS FOR ORDERED FOOD PREFERENCES ACCOMPANYING SYMPTOMATIC INPUTS,” each of which are incorporated by reference herein in their entirety.

### FIELD OF THE INVENTION

The present invention generally relates to the field of nourishment. In particular, the present invention is directed to methods and systems for ordered food preferences accompanying symptomatic inputs.

### BACKGROUND

Food element preferences can change over time. In addition, various features can affect food element preferences. Utilizing food preferences in combination with selecting food elements that minimize symptomatic inputs remain to be seen.

### SUMMARY OF THE DISCLOSURE

In an aspect, a system for generating food preference menu, the system comprising a computing device, the computing device designed and configured to receive a plurality of data sets from one or more data sources, classify the plurality of data sets into one or more user groups, wherein classifying the plurality of data sets includes identifying a plurality of data elements from the plurality of data sets and classifying the plurality of data sets into the one or more user groups as a function of the plurality of data elements, identify a food pattern of the plurality of data sets in the one or more user groups, generate a food preference menu as a function of the food pattern, wherein the food preference menu includes a nourishment strategy and update the food preference menu as a function of newly collected data.

In an aspect, a method for generating food preference menu, the method comprising receiving, using a computing device, a plurality of data sets from one or more data sources, classifying, using the computing device, the plurality of data sets into one or more user groups, wherein classifying the plurality of data sets includes identifying a plurality of data elements from the plurality of data sets and classifying the plurality of data sets into the one or more user groups as a function of the plurality of data elements, identifying, using the computing device, a food pattern of the plurality of data sets in the one or more user groups, generating, using the computing device, a food preference menu as a function of the food pattern, wherein the food preference menu includes a nourishment strategy and updating, using the computing device, the food preference menu as a function of newly collected data.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 illustrates a block diagram of an exemplary system for generating food preference menu;

FIG. 2 illustrates a block diagram of an exemplary embodiment of a symptomatic database;

FIG. 3 illustrates a diagrammatic representation of a food element list;

FIG. 4 illustrates a block diagram of an exemplary chatbot system;

FIG. 5 illustrates a block diagram of an exemplary embodiment of a machine-learning module;

FIG. 6 illustrates a diagram of an exemplary nodal network;

FIG. 7 illustrates a block diagram of an exemplary node;

FIG. 8 is a process flow diagram illustrating an exemplary embodiment of a method of ordered food preferences accompanying symptomatic inputs;

FIG. 9 illustrates a flow diagram of an exemplary method for generating food preference menu; and

FIG. 10 is a block diagram of a computing system that can be used to implement any one or more of the methodologies disclosed herein and any one or more portions thereof.

The drawings are not necessarily to scale and may be illustrated by phantom lines, diagrammatic representations, and fragmentary views. In certain instances, details that are not necessary for an understanding of the embodiments or that render other details difficult to perceive may have been omitted.

### DETAILED DESCRIPTION

At a high level, aspects of the present disclosure are directed to systems and methods for ordered food preferences accompanying symptomatic inputs. In an embodiment, symptomatic input is utilized to generate a food preference menu. Genetic food preferences are utilized in combination with a machine-learning process to identify food preferences, and rank food elements utilizing such information.

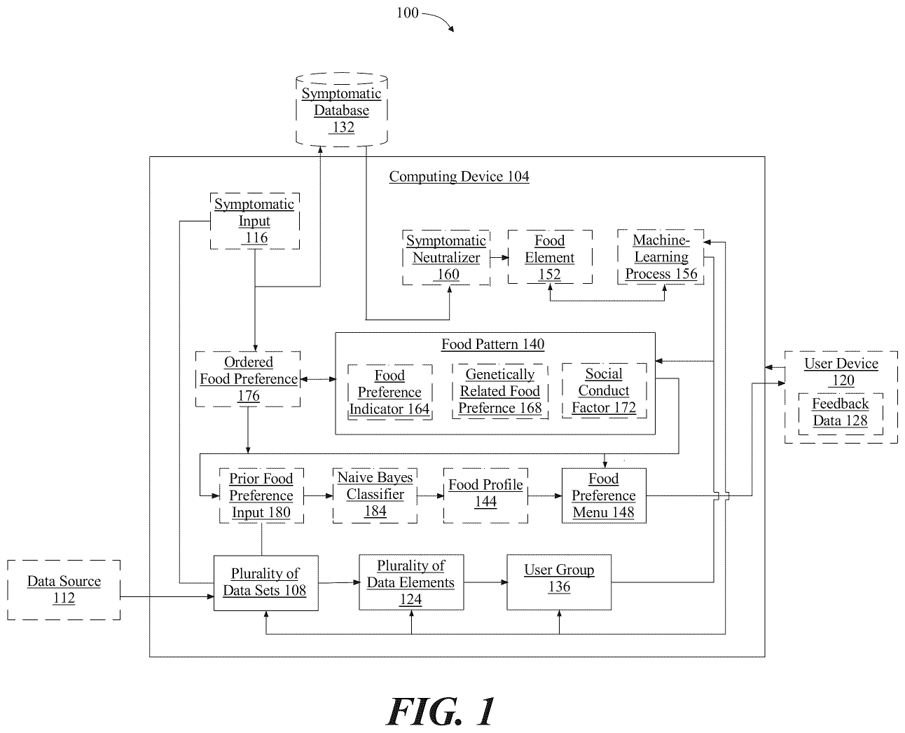

Referring now to FIG. 1, an exemplary embodiment of a system 100 for ordered food preferences accompanying symptomatic inputs is illustrated. System 100 includes a computing device 104. Computing device 104 may include any computing device 104 as described in this disclosure, including without limitation a microcontroller, microprocessor, digital signal processor (DSP) and/or system on a chip (SoC) as described in this disclosure. Computing device 104 may include, be included in, and/or connect with a mobile device such as a mobile telephone or smartphone. Computing device 104 may include a single computing device 104 operating independently or may include two or more computing device 104 operating in concert, in parallel, sequentially or the like; two or more computing devices 104 may be included together in a single computing device 104 or in two or more computing devices 104. Computing device 104 may interface or connect with one or more additional devices as described below in further detail via a network interface device. Network interface device may be utilized for connecting computing device 104 to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an association, a building, a campus or other relatively small geographic space), a telephone network, a data network associated with a telephone/voice provider (e.g., a mobile communications provider data and/or voice network), a direct connection between two computing devices 104, and any combinations thereof. A network may employ a wired and/or a wireless mode of communication. In general, any network topology may be used. Information (e.g., data, software etc.) may be transmitted to and/or from a computer and/or a computing device 104. Computing device 104 may include but is not limited to, for example, a computing device 104 or cluster of computing devices 104 in a first position and a second computing device 104 or cluster of computing devices 104 in a second position. Computing device 104 may include one or more computing devices 104 dedicated to data storage, security, dispersal of traffic for load balancing, and the like. Computing device 104 may distribute one or more computing tasks as described below across a plurality of computing devices 104 of computing device 104, which may operate in parallel, in series, redundantly, or in any other manner used for dispersal of tasks or memory between computing devices 104. Computing device 104 may be implemented using a “shared nothing” architecture in which data is cached at the operative, in an embodiment, this may enable scalability of system 100 and/or computing device 104.

Continuing to refer to FIG. 1, computing device 104 may be designed and/or configured to perform any method, method step, or sequence of method steps in any embodiment described in this disclosure, in any order and with any degree of repetition. For instance, computing device 104 may be configured to perform a single step or sequence recurrently until a desired or commanded outcome is achieved; repetition of a step or a sequence of steps may be performed iteratively and/or recursively using outputs of previous repetitions as inputs to subsequent repetitions, assembling inputs and/or outputs of repetitions to produce an aggregate result, reduction or decrement of one or more variables such as global variables, and/or division of a larger processing task into a set of iteratively addressed smaller processing tasks. Computing device 104 may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

With continued reference to FIG. 1, computing device 104 is configured to receive a plurality of data sets 108 from one or more data sources 112. For the purposes of this disclosure, a “plurality of data sets” is a collection or a group of multiple datasets. In some embodiments, plurality of data sets 108 may be related to a user. Exemplary data sets 108 may include symptomatic input 116, symptomatic complaint, diagnosis, element of previous physical data, demographic information, prior food preference input, or the like as described below. Exemplary data sets 108 may include document, text, icon, image, video, or the like. Exemplary data sets 108 may include symptomatic input 116, symptomatic complaint, diagnosis, element of previous physical data, demographic information, prior food preference input, or the like as described below. For the purposes of this disclosure, a “user” is any individual, group, company, organization or entity. As a non-limiting example, a user may include one person, a family, teams in companies, or the like.

With continued reference to FIG. 1, for the purposes of this disclosure, a “data source” is any place, system, tool, device or location from which user data originates. In some embodiments, data source 112 may include a user device 120. For the purposes of this disclosure, a “user device” is any device a user uses to input data. As a non-limiting example, user device 120 may include a laptop, desktop, tablet, mobile phone, smart phone, smart watch, kiosk, screen, smart headset, or things of the like. In some embodiments, user device 120 may include a user interface configured to receive inputs from user. In some embodiments, user may manually input any data such as but not limited to plurality of data sets 108, plurality of data elements 124, feedback data 128, or the like into apparatus 100 using user device 120. In some embodiments, user may have a capability to process, store or transmit any information independently. In some embodiments, one or more data sources 112 may include an application residing on user device 120. For the purposes of this disclosure, a “user interface” is a means by which a user and a computer system interact; for example through the use of input devices and software. A user interface may include a graphical user interface (GUI), command line interface (CLI), menu-driven user interface, touch user interface, voice user interface (VUI), form-based user interface, any combination thereof and the like. In some embodiments, user interface may operate on and/or be communicatively connected to a decentralized platform, metaverse, and/or a decentralized exchange platform associated with the user. For example, a user may interact with user interface in virtual reality. In some embodiments, a user may interact with the use interface using a computing device such as but not limited to user device 120, distinct from and communicatively connected to a computing device 104. In an embodiment, user interface may include a graphical user interface. A “graphical user interface,” as used herein, is a graphical form of user interface that allows users to interact with electronic devices. In some embodiments, GUI may include icons, menus, other visual indicators or representations (graphics), audio indicators such as primary notation, and display information and related user controls. A menu may contain a list of choices and may allow entities to select one from them. A menu bar may be displayed horizontally across the screen such as pull-down menu. When any option is clicked in this menu, then the pull-down menu may appear. A menu may include a context menu that appears only when the user performs a specific action. An example of this is pressing the right mouse button. When this is done, a menu may appear under the cursor. Files, programs, web pages and the like may be represented using a small picture in a graphical user interface. For example, links to decentralized platforms as described in this disclosure may be incorporated using icons. Using an icon may be a fast way to open documents, run programs etc. because clicking on them yields instant access.

With continued reference to FIG. 1, in some embodiments, data source 112 may include a database. In some embodiments, database may be implemented, without limitation, as a relational database, a key-value retrieval database such as a NOSQL database, or any other format or structure for use as a database that a person skilled in the art would recognize as suitable upon review of the entirety of this disclosure. Database may alternatively or additionally be implemented using a distributed data storage protocol and/or data structure, such as a distributed hash table or the like. Database may include a plurality of data entries and/or records as described above. Data entries in a database may be flagged with or linked to one or more additional elements of information, which may be reflected in data entry cells and/or in linked tables such as tables related by one or more indices in a relational database. Persons skilled in the art, upon reviewing the entirety of this disclosure, will be aware of various ways in which data entries in a database may store, retrieve, organize, and/or reflect data and/or records as used herein, as well as categories and/or populations of data consistently with this disclosure. In one or more embodiments, database may include inputted or calculated information and datum. As a non-limiting example, the datum history may include real-time and/or previous inputted data. In some embodiments, database may include instructions from a user, who may be an expert user, a past user in embodiments disclosed herein, or the like, where the instructions may include examples of the data related to user or plurality of data sets 108.

With continued reference to FIG. 1, in some embodiments, database may include keywords. As used in this disclosure, a “keyword” is an element of word or syntax used to identify and/or match elements to each other. For example, without limitation, the keyword may include user's name in the instance that user is looking for data related to user. In another non-limiting example, the keyword may include the name of project in the instance that user is looking for data related to a specific project.

With continued reference to FIG. 1, in some embodiments, database may include a symptomatic database 132. In some embodiments, data source 112 may include symptomatic database 132. As used in this disclosure, “symptomatic database” is a data structure configured to store data associated with user or a plurality of data sets. As a non-limiting example, symptomatic database 132 may store a plurality of data sets 108, plurality of data elements 124, user group 136, food pattern 140, feedback data 128, food profile 144, food preference menu 148, food element 152, input and/or output of machine-learning models, and the like. In one or more embodiments, symptomatic database 132 may include inputted or calculated information and datum related to user or a plurality of data sets 108. In some embodiments, a datum history may be stored in symptomatic database 132. As a non-limiting example, the datum history may include real-time and/or previously input data to computing device 104 related to user. As a non-limiting example, symptomatic database 132 may include instructions from a user, who may be an expert user, a past user in embodiments disclosed herein, or the like, where the instructions may include examples of the data related to user or a plurality of data sets 108. Symptomatic database 132 disclosed herein is further described below.

With continued reference to FIG. 1, in some embodiments, symptomatic database 132 or any database may be communicatively connected with computing device 104. For example, and without limitation, in some cases, symptomatic database 132 may be local to computing device 104. In another example, and without limitation, symptomatic database 132 may be remote to computing device 104 and communicative with computing device 104 by way of one or more networks. The network may include, but is not limited to, a cloud network, a mesh network, and the like. By way of example, a “cloud-based” system can refer to a system which includes software and/or data which is stored, managed, and/or processed on a network of remote servers hosted in the “cloud,” e.g., via the Internet, rather than on local severs or personal computers. A “mesh network” as used in this disclosure is a local network topology in which the infrastructure computing device 104 connect directly, dynamically, and non-hierarchically to as many other computing devices as possible. A “network topology” as used in this disclosure is an arrangement of elements of a communication network. The network may use an immutable sequential listing to securely store symptomatic database 132. An “immutable sequential listing,” as used in this disclosure, is a data structure that places data entries in a fixed sequential arrangement, such as a temporal sequence of entries and/or blocks thereof, where the sequential arrangement, once established, cannot be altered or reordered. An immutable sequential listing may be, include and/or implement an immutable ledger, where data entries that have been posted to the immutable sequential listing cannot be altered.

With continued reference to FIG. 1, in some embodiments, data source 112 may include application programming interface (API). As used herein, an “application programming interface” is a set of functions that allow applications to access data and interact with external software components, operating systems, or microdevices, such as another web application or computing device. In some embodiments, data source 112 may include cookies. For the purposes of this disclosure, “cookies” are small text files stored on a user's device that contain information about their browsing activities. In some embodiments, computing device 104 may track a user's web browsing using cookies of data source 112. In some embodiments, a plurality of data sets 108 may include user's web browsing history. As a non-limiting example, user's time usage can be obtained by leveraging cookies that track entity's interactions on a web page or application. For example, and without limitation, when a user visits a website or uses an online service, cookies may capture and store data. In a non-limiting example, cookies may capture and store the timestamp of a user's visit, the duration of user's session, the specific pages user accessed, or the like.

With reference to FIG. 1, in some embodiments, data source 112 may include web crawler. A “web crawler,” as used herein, is a program that systematically browses the internet for the purpose of Web indexing. The web crawler may be seeded with platform URLs, wherein the crawler may then visit the next related URL, retrieve the content, index the content, and/or measures the relevance of the content to the topic of interest. In some embodiments, computing device 104 may generate web crawler to scrape a plurality of data sets 108 from user's website. As a non-limiting example, computing device 104 may obtain user's web browsing history or pattern using web crawler. The web crawler may be seeded and/or trained with a reputable website to begin the search. Web crawler may be generated by computing device 104. In some embodiments, web crawler may be trained with information received from user through a user interface. In some embodiments, web crawler may be configured to generate a web query. A web query may include search criteria received from user. For example, user may submit a plurality of websites for web crawler to search to a plurality of data sets 108. Additionally, web crawler function may be configured to search for and/or detect one or more data patterns. A “data pattern,” as used in this disclosure, is any repeating forms of information. In some embodiments, web crawler may be configured to determine the relevancy of a data pattern. Relevancy may be determined by a relevancy score. A relevancy score may be automatically generated by computing device 104, received from a machine-learning model, and/or received from user. In some embodiments, a relevancy score may include a range of numerical values that may correspond to a relevancy strength of data received from a web crawler function. As a non-limiting example, a web crawler function may search the Internet for a plurality of data sets 108 related to user.

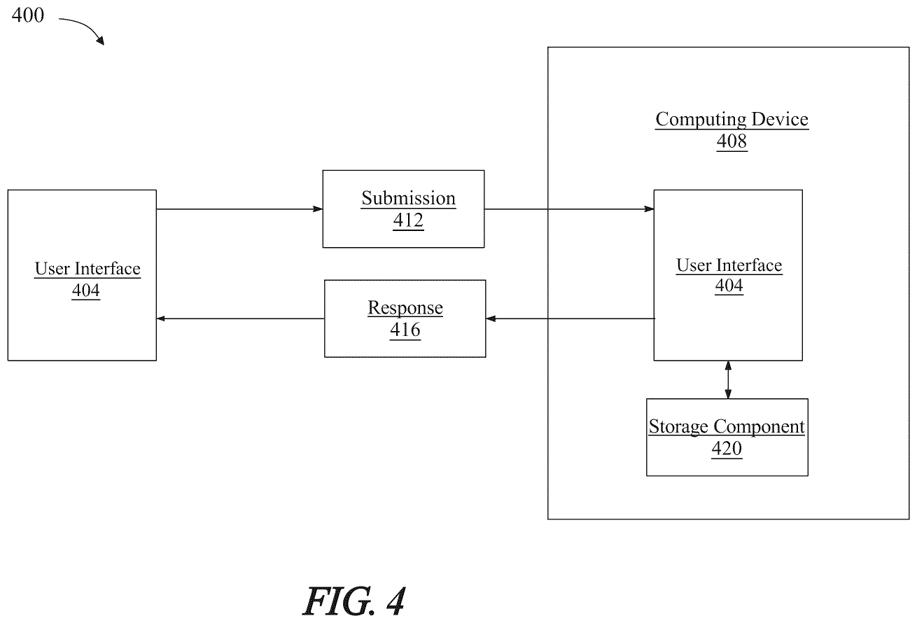

With continued reference to FIG. 1, in some embodiments, data source 112 may include a chatbot. For the purposes of this disclosure, “chatbot” is an artificial intelligence (AI) program designed to simulate human conversation or interaction through text, voice-based or image-based communication. Chatbot disclosed herein is further described with respect to FIG. 4. In a non-limiting example, computing device 104 may obtain a plurality of data sets 108 from user using chatbot. For example, and without limitation, a plurality of data sets 108 obtained using chatbot may include a question, response, statement, or the like input by user, question, response, statement, or the like generated for user. In some embodiments, chatbot may include generative artificial intelligence (AI), large language model (LLM), or the like. In some embodiments, data source 112 may include a secure communication channel interface. In some embodiments, computing device 104 may be configured to establish a secure communication channel interface between a user device 120 and computing device 104. A “secure communication channel interface,” as used in this disclosure, is a communication medium within an interface. A secure communication channel interface may include an application, script, and/or program capable of providing a means of communication between at least two parties, including any oral and/or written forms of communication. A secure communication channel interface may allow computing device 104 to interface with electronic devices through graphical icons, audio indicators including primary notation, text based user interfaces, typed command labels, text navigation, and the like. A secure communication channel interface may include slides or other commands that may allow entity to select one or more options. A secure communication channel interface may include free form textual entries, where a user may type in a prompt, response and/or message. In some embodiments, computing device 104 may be configured to receive prompt from user device 120 using secure communication channel interface. A secure communication channel interface may include a display interface. Display interface may include a form or other graphical element having display fields, where one or more elements of information may be displayed. Display interface may display data output fields including text, images, or the like containing one or more messages. A secure communication channel interface may include data input fields such as text entry windows, drop-down lists, buttons, checkboxes, radio buttons, sliders, links, or any other data input interface that may capture user interaction as may occur to persons skilled in the art upon reviewing the entirety of this disclosure.

With continued reference to FIG. 1, in some embodiments, plurality of data sets 108 may include a symptomatic input 116 relating to a user. A “symptomatic input,” as used in this disclosure, is a description of any physical and/or mental feature of a user that may indicate a condition and/or disease. A symptomatic input 116 includes a description of a symptomatic complaint. A “symptomatic complaint,” as used in this disclosure, is a description of any symptom a user has previously or is currently experiencing. A symptomatic complaint may include a symptom of a disease that a user experiences, such as a user who experiences joint pain and fatigue from rheumatoid arthritis. In yet another non-limiting example, a symptomatic complaint may contain a description of symptoms that a user who was previously diagnosed with fibroids experiences which includes heavy menstrual bleeding, pelvic pressure, and frequent urination. A symptomatic input may include a subjective symptom such as tiredness. A symptomatic input 116 may include an objective symptom such as a cough. A symptomatic input 116 may indicate a physiological state of a user, such as a pregnant female who complains of morning sickness. A symptomatic input 116 may include a brief acute symptom, such as a back spasm that comes on suddenly. A symptomatic input 116 may describe a chronic symptom, such as a dry cough and chest congestion that occurs every morning upon waking. A symptomatic complaint may include a description of one or more general symptoms that affect the entire body such as fever, malaise, anorexia, and weight loss.

With continued reference to FIG. 1, a symptomatic complaint may include a diagnosis. A “diagnosis,” as used in this disclosure, is a disease, syndrome, condition, disorder, sickness, ailment, and the like identified by a professional. A “professional,” as used in this disclosure, is any person with valid credentials and/or certifications to provide wellness services to natural persons. A professional may include a physician, a dentist, a nurse, a chiropractor, an optometrist, a physical therapist, an occupational therapist, a dietician, a nurse practitioner, a psychologist, a licensed professional counselor, a licensed marriage and/or family therapist, a pharmacist, a speech therapist and the like. A diagnosis may include a current condition that a user suffers from, such as ulcerative colitis. A diagnosis may include an impending condition that a user may be at danger of developing due to one or more features, such as heart disease or Type 2 Diabetes Mellitus. A diagnosis may include a condition that was cured and/or resolved, such as an ear infection or a meningitis. A diagnosis may be self-reported by a user, such as a user who self-reports a previous diagnosis of hypertension that a user is currently treating with a drug. A symptomatic input 116 may include an element of previous physical data. An “element of previous physical data,” as used in this disclosure, is any previous therapeutic and/or wellness information unique to a user. An element of previous physical data may include demographic information that may include a user's name, gender, age, birthday, occupation, family structure, living arrangements, marital status, and the like. An element of previous physical data may include information regarding any information regarding symptomatic complaints regarding specific body systems such as the head, eyes, ears, nose, and throat (HEENT), cardiovascular, respiratory, gastrointestinal, genitourinary, integumentary, musculoskeletal, endocrine, nervous system, mental, and the like. An element of previous physical data may relate to information describing a user's social well-being and social life, family history, mental or emotional illness or stresses, detrimental or beneficial habits, social life, smoking or exercise habits, educational level, previous surgical history, previous procedures, culture, sexuality, nutrition, spirituality, and the like. An element of previous physical data may relate to past wellness history such as allergies, serious or chronic illnesses, recent hospitalizations, recent surgical procedures, current drugs, alcohol consumption, marijuana use and the like. An element of previous physical data may be received based on one or more questions and/or self-assessments completed by a user.

With continued reference to FIG. 1, information pertaining to a symptomatic input 116 may be stored within symptomatic database 132. Symptomatic database 132 may be implemented, without limitation, as a relational database, a key-value retrieval datastore such as a NOSQL database, or any other format or structure for use as a datastore that a person skilled in the art would recognize as suitable upon review of the entirety of this disclosure.

With continued reference to FIG. 1, computing device 104 may receive a symptomatic input 116 relating to a user from a user device 120 operated by a user. A user device 120 may include without limitation, a display in communication with computing device 104, where a display may include any display as described herein. A user device 120 may include an additional computing device, such as a mobile device, laptop, desktop, computer, and the like.

With continued reference to FIG. 1, computing device 104 may be configured to generate a machine-learning process, wherein the machine-learning process is trained using a first training set relating symptomatic inputs to symptomatic neutralizers. A “machine-learning process,” as used in this disclosure, is a process that automatedly uses a body of data known as “training data” and/or a “training set” to generate an algorithm that will be performed by computing device 104 and/or a module to produce outputs given data provided as inputs; this is in contrast to a non-machine learning software program where the commands to be executed are determined in advance by a user and written in a programming language. “Training data,” as used in this disclosure, is a set of examples that contain pairs of an input and a corresponding output, which are used to model relationships between two or more categories of data elements. Training data may be formatted to include labels, for instance by associating data elements with one or more descriptors corresponding to categories of data elements. Training data may not contain labels; training data may not be formatted to include labels. A machine-learning process 156 may include calculating one or more machine-learning algorithms and/or producing one or more machine-learning models. A machine-learning process 156 may include a supervised machine-learning process 156 that applies learned associations from the past to new data using labeled training data to predict future events. A supervised machine-learning process 156 produces an inferred function to make predictions about output values. A supervised machine-learning process 156 may include for example, active learning, classification, regression, and/or similarity learning. A machine-learning process 156 may include an unsupervised machine-learning process 156 where training data utilized to train the unsupervised machine-learning process 156 may not be classified or labeled. An unsupervised machine-learning process 156 may infer a function to describe a hidden structure from unlabeled data. An unsupervised machine-learning process 156 may include for example, clustering, anomaly detection, neural networks, latent variable models, and the like. A machine-learning process 156 may include a semi-supervised machine-learning process 156 that may utilize a combination of both labeled and unlabeled training data. A semi-supervised machine-learning process 156 may include generative models, low density separation, graph-based methods, heuristic approaches, and the like. A machine-learning process 156 may be implemented as any machine-learning process, including for instance, and without limitation, as described in U.S. Nonprovisional application Ser. No. 16/375,303, filed on Apr. 4, 2019, and entitled “SYSTEMS AND METHODS FOR GENERATING ALIMENTARY INSTRUCTION SETS BASED ON VIBRANT CONSTITUTIONAL GUIDANCE,” the entirety of which is incorporated herein by reference.

With continued reference to FIG. 1, training data utilized to train a machine-learning process 156 may be obtained from records of previous iterations of a machine-learning process 156, user inputs and/or questionnaire responses, expert inputs, open source platforms and the like. Computing device 104 trains a machine-learning process 156 including any machine-learning algorithm and/or any machine-learning model utilizing a first training set relating symptomatic inputs to food preferences.

With continued reference to FIG. 1, computing device 104 may be configured to identify a symptomatic neutralizer 160 based on a symptomatic input using a machine-learning process. A “food element,” as used in this disclosure, is any food, beverage, drink, snack, nutritional supplement, and the like intended for consumption by a human being. A “symptomatic neutralizer,” as used in this disclosure, is any indication as to how much or how little a food element 152 helps alleviate or exacerbate a symptomatic input 116. For instance and without limitation, a food element 152 such as organ meats may exacerbate a condition of gout, while a food such as sauerkraut may alleviate symptoms associated with a condition such as irritable bowel syndrome. A symptomatic neutralizer 160 may be expressed as a quantitative datum, containing a numerical score that indicates how much or how little a food element alleviates or exacerbates a symptomatic input 116. A symptomatic neutralizer 160 may be expressed on a numerical score from 0 to 100 for example, where a score of 0 may indicate a food element that exacerbates a symptomatic input 116, while a score of 100 may indicate a food element that alleviates a symptomatic input. For instance and without limitation, a symptomatic neutralizer 160 for a food element such as tomatoes may contain a quantitative datum containing a numerical score of 17 for exacerbating symptoms of a stomach ulcer, while a symptomatic neutralizer 160 for a food element such as honey may contain a quantitative datum containing a numerical score of 77 for alleviating symptoms of a stomach ulcer. Computing device 104 generates a symptomatic neutralizer 160 utilizing one or more regression processes, including any of the regression processes as described herein.

With continued reference to FIG. 1, computing device 104 is configured to classify a plurality of data sets 108 into one or more user groups 136. For the purposes of this disclosure, a “user group” is a set of associative data sets pertaining to associative users. As a non-limiting example, user group 136 may include first user, second user, third user group, or the like. As another non-limiting example, user group 136 may include first family, second family, third family group, or the like. In a non-limiting example, one user group 136 may include a plurality of users associated to each other. For example, and without limitation, first user group may include a plurality of users who are a blood family. For another example, and without limitation, second user group may include a plurality of users who are in the same team of a company. User group 136 may include a plurality data sets 108 or plurality of data elements 124 related to a plurality of users in user group 136. In some embodiments, user group 136 may be stored in symptomatic database 132. In some embodiments, user group 136 may be retrieved from symptomatic database 132.

With continued reference to FIG. 1, computing device 104 is configured to classify a plurality of data sets 108 into one or more user groups 136. In some embodiments, classifying a plurality of data sets 108 into one or more user groups 136 includes identifying a plurality of data elements 124 pertaining to a plurality of users from a plurality of data sets 108 and classify the plurality of data sets 108 to one or more user groups 136 as a function of the plurality of data elements 124. For the purposes of this disclosure, a “user group” is a set of associative data sets pertaining to associative users. As a non-limiting example, user group 136 may include first user, second user, third user group, or the like. As another non-limiting example, user group 136 may include first family, second family, third family group, or the like. In a non-limiting example, one user group 136 may include a plurality of users associated to each other. For example, and without limitation, first user group may include a plurality of users who are a blood family. For another example, and without limitation, second user group may include a plurality of users who are in the same team of a company. User group 136 may include a plurality data sets 108 or plurality of data elements 124 related to a plurality of users in user group 136. In a non-limiting example, computing device 104 may be configured to classify data sets 108 into a first user group that is related to a specific team when includes data elements 124 includes information related to the specific team of a company. In some embodiments, user group 136 may be stored in symptomatic database 132. In some embodiments, user group 136 may be retrieved from symptomatic database 132. For the purposes of this disclosure, a “plurality of data elements” is elements of data within a dataset or a collection. Exemplary data elements 124 may include keywords, labels of image, or the like. As a non-limiting example, data elements 124 may include family last name, position in company, name of team, name of food, appetite size, financial budget, or the like. In some embodiments, data elements 124 may be stored in symptomatic database 132. In some embodiments, data elements 124 may be retrieved from symptomatic database 132.

With continued reference to FIG. 1, in some embodiments, computing device 104 may analyze a plurality of data sets 108 or document to find a plurality of data elements 124 using optical character recognition (OCR). For the purposes of this disclosure, “optical character recognition” is a technology that enables the recognition and conversion of printed or written text into machine-encoded text. In some cases, computing device 104 may be configured to recognize a keyword using the OCR to find a plurality of data elements 124. As used in this disclosure, a “keyword” is an element of word or syntax used to identify and/or match elements to each other. In some cases, computing device 104 may transcribe much or even substantially all data sets 108.

With continued reference to FIG. 1, in some embodiments, optical character recognition or optical character reader (OCR) may include automatic conversion of images of written (e.g., typed, handwritten or printed text) into machine-encoded text. In some cases, recognition of a keyword from a plurality of data sets 108 may include one or more processes, including without limitation optical character recognition (OCR), optical word recognition, intelligent character recognition, intelligent word recognition, and the like. In some cases, OCR may recognize written text, one glyph or character at a time. In some cases, optical word recognition may recognize written text, one word at a time, for example, for languages that use a space as a word divider. In some cases, intelligent character recognition (ICR) may recognize written text one glyph or character at a time, for instance by employing machine-learning processes. In some cases, intelligent word recognition (IWR) may recognize written text, one word at a time, for instance by employing machine-learning processes.

With continued reference to FIG. 1, in some cases, OCR may be an “offline” process, which analyses a static document or image frame. In some cases, handwriting movement analysis can be used as input to handwriting recognition. For example, instead of merely using shapes of glyphs and words, this technique may capture motions, such as the order in which segments are drawn, the direction, and the pattern of putting the pen down and lifting it. This additional information may make handwriting recognition more accurate. In some cases, this technology may be referred to as “online” character recognition, dynamic character recognition, real-time character recognition, and intelligent character recognition.

With continued reference to FIG. 1, in some cases, OCR processes may employ pre-processing of a plurality of data sets 108. Pre-processing process may include without limitation de-skew, de-speckle, binarization, line removal, layout analysis or “zoning,” line and word detection, script recognition, character isolation or “segmentation,” and normalization. In some cases, a de-skew process may include applying a transform (e.g., homography or affine transform) to a plurality of data sets 108 to align text. In some cases, a de-speckle process may include removing positive and negative spots and/or smoothing edges. In some cases, a binarization process may include converting an image from color or greyscale to black-and-white (i.e., a binary image). Binarization may be performed as a simple way of separating text (or any other desired image component) from a background of image component. In some cases, binarization may be required for example if an employed OCR algorithm only works on binary images. In some cases, a line removal process may include removal of non-glyph or non-character imagery (e.g., boxes and lines). In some cases, a layout analysis or “zoning” process may identify columns, paragraphs, captions, and the like as distinct blocks. In some cases, a line and word detection process may establish a baseline for word and character shapes and separate words, if necessary. In some cases, a script recognition process may, for example in multilingual documents, identify script allowing an appropriate OCR algorithm to be selected. In some cases, a character isolation or “segmentation” process may separate signal characters, for example character-based OCR algorithms. In some cases, a normalization process may normalize aspect ratio and/or scale of image component.

With continued reference to FIG. 1, in some embodiments an OCR process may include an OCR algorithm. Exemplary OCR algorithms include matrix matching process and/or feature extraction processes. Matrix matching may involve comparing an image to a stored glyph on a pixel-by-pixel basis. In some case, matrix matching may also be known as “pattern matching,” “pattern recognition,” and/or “image correlation.” Matrix matching may rely on an input glyph being correctly isolated from the rest of the image component. Matrix matching may also rely on a stored glyph being in a similar font and at a same scale as input glyph. Matrix matching may work best with typewritten text.

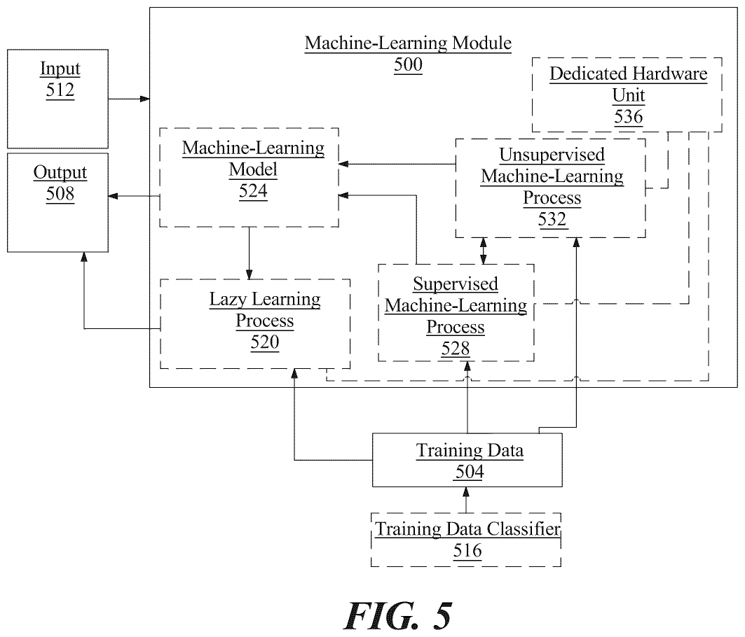

With continued reference to FIG. 1, in some embodiments, an OCR process may include a feature extraction process. In some cases, feature extraction may decompose a glyph into a feature. Exemplary non-limiting features may include corners, edges, lines, closed loops, line direction, line intersections, and the like. In some cases, feature extraction may reduce dimensionality of representation and may make the recognition process computationally more efficient. In some cases, extracted feature may be compared with an abstract vector-like representation of a character, which might reduce to one or more glyph prototypes. General techniques of feature detection in computer vision are applicable to this type of OCR. In some embodiments, machine-learning processes like nearest neighbor classifiers (e.g., k-nearest neighbors algorithm) may be used to compare image features with stored glyph features and choose a nearest match. OCR may employ any machine-learning process described in this disclosure, for example machine-learning processes described with reference to FIG. 5. Exemplary non-limiting OCR software may include Cuneiform and Tesseract. Cuneiform may include a multi-language, open-source optical character recognition system originally developed by Cognitive Technologies of Moscow, Russia. Tesseract may include free OCR software originally developed by Hewlett-Packard of Palo Alto, California, United States.

With continued reference to FIG. 1, in some cases, OCR may employ a two-pass approach to character recognition. A first pass may try to recognize a character. Each character that is satisfactory may be passed to an adaptive classifier as training data. The adaptive classifier then may get a chance to recognize characters more accurately as it further analyzes a plurality of data sets 108. Since the adaptive classifier may have learned something useful a little too late to recognize characters on the first pass, a second pass may be run over a plurality of data sets 108. Second pass may include adaptive recognition and use characters recognized with high confidence on the first pass to recognize better remaining characters on the second pass. In some cases, two-pass approach may be advantageous for unusual fonts or low-quality image components where visual verbal content may be distorted. Another exemplary OCR software tool may include OCRopus. OCRopus development is led by German Research Centre for Artificial Intelligence in Kaiserslautern, Germany. In some cases, OCR software may employ neural networks.

With continued reference to FIG. 1, in some cases, OCR may include post-processing. For example, OCR accuracy may be increased, in some cases, if output is constrained by a lexicon. A lexicon may include a list or set of words that are allowed to occur in a document. In some cases, a lexicon may include, for instance, all the words in the English language, or a more technical lexicon for a specific field. In some cases, an output stream may be a plain text stream or file of characters. In some cases, an OCR process may preserve an original layout of visual verbal content. In some cases, near-neighbor analysis can make use of co-occurrence frequencies to correct errors, by noting that certain words are often seen together. For example, “Washington, D.C.” is generally far more common in English than “Washington DOC.” In some cases, an OCR process may make us of a priori knowledge of grammar for a language being recognized. For example, grammar rules may be used to help determine if a word is likely to be a verb or a noun. Distance conceptualization may be employed for recognition and classification. For example, a Levenshtein distance algorithm may be used in OCR post-processing to further optimize results.

With continued reference to FIG. 1, in some embodiments, computing device 104 may be configured to identify a plurality of data elements 124 of a plurality of data sets 108 using a machine vision system. For the purposes of this disclosure, a “machine vision system” is a type of technology that enables a computing device or processing module to inspect, evaluate and identify still or moving images. A machine vision system may make a determination about a scene, space, food and/or object (data elements 124) of image or video of a plurality of data sets 108. For example, in some cases, a machine vision system may be used for world modeling or registration of objects within a space. In some cases, registration may include image processing, such as without limitation object recognition, feature detection, edge/corner detection, and the like. Non-limiting example of feature detection may include scale invariant feature transform (SIFT), Canny edge detection, Shi Tomasi corner detection, and the like. In some cases, a machine vision process may operate image classification and segmentation models, such as without limitation by way of machine vision resource (e.g. OpenMV or TensorFlow Lite). A machine vision process may detect motion, for example by way of frame differencing algorithms. A machine vision process may detect markers, for example blob detection, object detection, face detection, and the like. In some cases, a machine vision process may perform eye tracking (i.e. gaze estimation). In some cases, a machine vision process may perform person detection, for example by way of a trained machine-learning model. In some cases, a machine vision process may perform motion detection (e.g. camera motion and/or object motion), for example by way of optical flow detection. In some cases, machine vision process may perform code (e.g. barcode) detection and decoding. In some cases, a machine vision process may additionally perform image capture and/or video recording.

With continued reference to FIG. 1, in some cases, registration may include one or more transformations to orient a camera frame (or an image or video stream) relative a three-dimensional coordinate system; exemplary transformations include without limitation, homography transforms and affine transforms. In an embodiment, registration of first frame to a coordinate system may be verified and/or corrected using object identification and/or computer vision. For instance, and without limitation, an initial registration to two dimensions, represented for instance as registration to the x and y coordinates, may be performed using a two-dimensional projection of points in three dimensions onto a first frame, however. A third dimension of registration, representing depth and/or a z axis, may be detected by comparison of two frames; for instance, where first frame includes a pair of frames captured using a pair of cameras (e.g. stereoscopic camera also referred to in this disclosure as stereo-camera), image recognition and/or edge detection software may be used to detect a pair of stereoscopic views of images of an object; two stereoscopic views may be compared to derive z-axis values of points on object permitting, for instance, derivation of further z-axis points within and/or around the object using interpolation. This may be repeated with multiple objects in field of view, including without limitation, any features of interest identified by a classifier and/or indicated by user. In an embodiment, x and y axes may be chosen to span a plane common to two cameras used for stereoscopic image capturing and/or an xy plane of a first frame; a result, x and y translational components and ϕ may be pre-populated in translational and rotational matrices, for affine transformation of coordinates of object, also as described above. Initial x and y coordinates and/or guesses at transformational matrices may alternatively or additionally be performed between first frame and second frame, as described above. For each point of a plurality of points on object and/or edge and/or edges of object as described above, x and y coordinates of a first stereoscopic frame may be populated, with an initial estimate of z coordinates based, for instance, on assumptions about object, such as an assumption that ground is substantially parallel to an xy plane as selected above. Z coordinates, and/or x, y, and z coordinates, registered using image capturing and/or object identification processes as described above may then be compared to coordinates predicted using initial guess at transformation matrices; an error function may be computed using by comparing the two sets of points, and new x, y, and/or z coordinates, may be iteratively estimated and compared until the error function drops below a threshold level.

With continued reference to FIG. 1, alternatively or additionally, identifying a shape (a plurality of data elements 124) in image or video of a plurality of data sets 108 may include classifying the shape (a plurality of data elements 124) in image or video of a plurality of data sets 108 to a label of a plurality of data elements 124 using an image classifier. In a non-limiting example, computing device 104 may be configured to generate image training data, train an image classifier using the image training data and determine a label of a plurality of data elements 124 using the trained image classifier. As a non-limiting example, image training data may include correlations between exemplary images or videos or exemplary data sets and exemplary labels of a plurality of data elements 124. In some embodiments, image classifier may be configured to determine which of a plurality of edge-detected shapes is closest to labels. Alternatively, identification of label of a plurality of data elements 124 may be performed without using computer vision and/or classification; for instance, identifying a plurality of data elements 124 may further include receiving, from a user, an identification of a plurality of data elements 124 of a plurality of data sets 108. In some embodiments, image training data may be received from one or more users, symptomatic database 132, external computing devices, and/or previous iterations of processing. As a non-limiting example, image training data may include instructions from a user, who may be an expert user, a past user in embodiments disclosed herein, or the like, which may be stored in memory and/or stored in symptomatic database 132, where the instructions may include labeling of training examples. In some embodiments, image training data may be updated iteratively through a feedback loop. As a non-limiting example, image training data may be updated iteratively through a feedback loop as a function of newly collected a plurality of data sets 108, a plurality of data elements 124, or the like. In some embodiments, computing device 104 may update image training data and retrain image classifier using the updated training data and determine a label of a plurality of data elements 124 using the retrained image classifier.

With continued reference to FIG. 1, in some embodiments, data elements 124 may include keywords. In some embodiments, computing device 104 may identify keywords (data elements 124) from a plurality of data sets 108 using a language processing module. Language processing module may be configured to extract, from one or more documents or plurality of data sets 108, one or more words. One or more words may include, without limitation, strings of one or more characters, including without limitation any sequence or sequences of letters, numbers, punctuation, diacritic marks, engineering symbols, geometric dimensioning and tolerancing (GD&T) symbols, chemical symbols and formulas, spaces, whitespace, and other symbols, including any symbols usable as textual data as described above. Textual data may be parsed into tokens, which may include a simple word (sequence of letters separated by whitespace) or more generally a sequence of characters as described previously. The term “token,” as used herein, refers to any smaller, individual groupings of text from a larger source of text; tokens may be broken up by word, pair of words, sentence, or other delimitation. These tokens may in turn be parsed in various ways. Textual data may be parsed into words or sequences of words, which may be considered words as well. Textual data may be parsed into “n-grams,” where all sequences of n consecutive characters are considered. Any or all possible sequences of tokens or words may be stored as “chains,” for example for use as a Markov chain or Hidden Markov Model.

With continued reference to FIG. 1, language processing module may operate to produce a language processing model. Language processing model may include a program automatically generated by computing device 104 and/or language processing module to produce associations between one or more words extracted from at least a document and detect associations, including without limitation mathematical associations, between such words. Associations between language elements, where language elements include for purposes herein extracted words, relationships of such categories to other such term may include, without limitation, mathematical associations, including without limitation statistical correlations between any language element and any other language element and/or language elements. Statistical correlations and/or mathematical associations may include probabilistic formulas or relationships indicating, for instance, a likelihood that a given extracted word indicates a given category of semantic meaning. As a further example, statistical correlations and/or mathematical associations may include probabilistic formulas or relationships indicating a positive and/or negative association between at least an extracted word and/or a given semantic meaning; positive or negative indication may include an indication that a given document is or is not indicating a category semantic meaning. Whether a phrase, sentence, word, or other textual element in a document or corpus of documents constitutes a positive or negative indicator may be determined, in an embodiment, by mathematical associations between detected words, comparisons to phrases and/or words indicating positive and/or negative indicators that are stored in memory at computing device, or the like.

With continued reference to FIG. 1, language processing module and/or diagnostic engine may generate the language processing model by any suitable method, including without limitation a natural language processing classification algorithm; language processing model may include a natural language process classification model that enumerates and/or derives statistical relationships between input terms and output terms. Algorithm to generate language processing model may include a stochastic gradient descent algorithm, which may include a method that iteratively optimizes an objective function, such as an objective function representing a statistical estimation of relationships between terms, including relationships between input terms and output terms, in the form of a sum of relationships to be estimated. In an alternative or additional approach, sequential tokens may be modeled as chains, serving as the observations in a Hidden Markov Model (HMM). HMMs, as used herein, are statistical models with inference algorithms that that may be applied to the models. In such models, a hidden state to be estimated may include an association between an extracted words, phrases, and/or other semantic units. There may be a finite number of categories to which an extracted word may pertain; an HMM inference algorithm, such as the forward-backward algorithm or the Viterbi algorithm, may be used to estimate the most likely discrete state given a word or sequence of words. Language processing module may combine two or more approaches. For instance, and without limitation, machine-learning program may use a combination of Naive-Bayes (NB), Stochastic Gradient Descent (SGD), and parameter grid-searching classification techniques; the result may include a classification algorithm that returns ranked associations.

With continued reference to FIG. 1, generating language processing model may include generating a vector space, which may be a collection of vectors, defined as a set of mathematical objects that can be added together under an operation of addition following properties of associativity, commutativity, existence of an identity element, and existence of an inverse element for each vector, and can be multiplied by scalar values under an operation of scalar multiplication compatible with field multiplication, and that has an identity element is distributive with respect to vector addition, and is distributive with respect to field addition. Each vector in an n-dimensional vector space may be represented by an n-tuple of numerical values. Each unique extracted word and/or language element as described above may be represented by a vector of the vector space. In an embodiment, each unique extracted and/or other language element may be represented by a dimension of vector space; as a non-limiting example, each element of a vector may include a number representing an enumeration of co-occurrences of the word and/or language element represented by the vector with another word and/or language element. Vectors may be normalized, scaled according to relative frequencies of appearance and/or file sizes. In an embodiment associating language elements to one another as described above may include computing a degree of vector similarity between a vector representing each language element and a vector representing another language element; vector similarity may be measured according to any norm for proximity and/or similarity of two vectors, including without limitation cosine similarity, which measures the similarity of two vectors by evaluating the cosine of the angle between the vectors, which can be computed using a dot product of the two vectors divided by the lengths of the two vectors. Degree of similarity may include any other geometric measure of distance between vectors.

With continued reference to FIG. 1, language processing module may use a corpus of documents to generate associations between language elements in a language processing module, and diagnostic engine may then use such associations to analyze words extracted from one or more documents and determine that the one or more documents indicate significance of a category. In an embodiment, language module and/or computing device 104 may perform this analysis using a selected set of significant documents, such as documents identified by one or more experts as representing good information; experts may identify or enter such documents via graphical user interface or may communicate identities of significant documents according to any other suitable method of electronic communication, or by providing such identity to other persons who may enter such identifications into computing device 104. Documents may be entered into a computing device by being uploaded by an expert or other persons using, without limitation, file transfer protocol (FTP) or other suitable methods for transmission and/or upload of documents; alternatively or additionally, where a document is identified by a citation, a uniform resource identifier (URI), uniform resource locator (URL) or other datum permitting unambiguous identification of the document, diagnostic engine may automatically obtain the document using such an identifier, for instance by submitting a request to a database or compendium of documents such as JSTOR as provided by Ithaka Harbors, Inc. of New York.

With continued reference to FIG. 1, computing device 104 may be configured to classify a plurality of data sets 108 and/or data elements 124 into one or more user groups 136 using a group classifier. As used in this disclosure, a “group classifier” is a machine-learning model, such as a mathematical model, neural net, or program generated by a machine learning algorithm known as a “classification algorithm,” that sorts a plurality of data sets related inputs into categories or bins of data, outputting one or more user groups associated therewith. In some embodiments, machine-learning process 156 may include group classifier. The group classifier disclosed herein may be consistent with a classifier disclosed with respect to FIG. 5. In some embodiments, computing device 104 may be configured to generate group training data. In some embodiments, group training data may include correlations between exemplary data elements or exemplary data sets and exemplary user group. In a non-limiting example, computing device 104 may be configured to classify data sets 108 into a first user group that is related to a specific team that includes information related to the specific team of a company (data elements 124) using group classifier trained or retrained with group training data. In some embodiments, group classifier may be trained with group training data and computing device 104 may classify data elements 124 to user group 136 using trained or retrained group classifier. The training data disclosed herein is further disclosed with respect to FIG. 5. In some embodiments, group training data may be stored in symptomatic database 132. In some embodiments, group training data may be received from one or more users, symptomatic database 132, external computing devices, and/or previous iterations of processing. As a non-limiting example, group training data may include instructions from a user, who may be an expert user, a past user in embodiments disclosed herein, or the like, which may be stored in memory and/or stored in symptomatic database 132, where the instructions may include labeling of training examples. In some embodiments, group training data may be iteratively updated through a feedback loop as a function of newly collected data such as but not limited to newly collected data elements 124, data sets 108, user group 136, outputs of one or more machine-learning models described in the entirety of this disclosure. As a non-limiting example, group training data may be iteratively updated through a feedback loop as a function of newly collected outputs of image classifier, language processing module, or the like.

With continued reference to FIG. 1, computing device 104 may be configured to generate a classifier (such as but not limited to group classifier, image classifier, or the like) using a Naïve Bayes classification algorithm. Naïve Bayes classification algorithm generates classifiers by assigning class labels to problem instances, represented as vectors of element values. Class labels are drawn from a finite set. Naïve Bayes classification algorithm may include generating a family of algorithms that assume that the value of a particular element is independent of the value of any other element, given a class variable. Naïve Bayes classification algorithm may be based on Bayes Theorem expressed as P(A/B)=P(B/A) P(A)÷P(B), where P(A/B) is the probability of hypothesis A given data B also known as posterior probability; P(B/A) is the probability of data B given that the hypothesis A was true; P(A) is the probability of hypothesis A being true regardless of data also known as prior probability of A; and P(B) is the probability of the data regardless of the hypothesis. A naïve Bayes algorithm may be generated by first transforming training data into a frequency table. Computing device 104 may then calculate a likelihood table by calculating probabilities of different data entries and classification labels. Computing device 104 may utilize a naïve Bayes equation to calculate a posterior probability for each class. A class containing the highest posterior probability is the outcome of prediction. Naïve Bayes classification algorithm may include a gaussian model that follows a normal distribution. Naïve Bayes classification algorithm may include a multinomial model that is used for discrete counts. Naïve Bayes classification algorithm may include a Bernoulli model that may be utilized when vectors are binary.

With continued reference to FIG. 1, computing device 104 may be configured to generate classifier (such as but not limited to group classifier, image classifier, or the like) using a K-nearest neighbors (KNN) algorithm. A “K-nearest neighbors algorithm” as used in this disclosure, includes a classification method that utilizes feature similarity to analyze how closely out-of-sample-features resemble training data to classify input data to one or more clusters and/or categories of features as represented in training data; this may be performed by representing both training data and input data in vector forms, and using one or more measures of vector similarity to identify classifications within training data, and to determine a classification of input data. K-nearest neighbors algorithm may include specifying a K-value, or a number directing the classifier to select the k most similar entries training data to a given sample, determining the most common classifier of the entries in the database 200, and classifying the known sample; this may be performed recursively and/or iteratively to generate a classifier that may be used to classify input data as further samples. For instance, an initial set of samples may be performed to cover an initial heuristic and/or “first guess” at an output and/or relationship, which may be seeded, without limitation, using expert input received according to any process as described herein. As a non-limiting example, an initial heuristic may include a ranking of associations between inputs and elements of training data. Heuristic may include selecting some number of highest-ranking associations and/or training data elements.

With continued reference to FIG. 1, generating k-nearest neighbors algorithm may generate a first vector output containing a data entry cluster, generating a second vector output containing an input data, and calculate the distance between the first vector output and the second vector output using any suitable norm such as cosine similarity, Euclidean distance measurement, or the like. Each vector output may be represented, without limitation, as an n-tuple of values, where n is at least two values. Each value of n-tuple of values may represent a measurement or other quantitative value associated with a given category of data, or attribute, examples of which are provided in further detail below; a vector may be represented, without limitation, in n-dimensional space using an axis per category of value represented in n-tuple of values, such that a vector has a geometric direction characterizing the relative quantities of attributes in the n-tuple as compared to each other. Two vectors may be considered equivalent where their directions, and/or the relative quantities of values within each vector as compared to each other, are the same; thus, as a non-limiting example, a vector represented as [5, 10, 15] may be treated as equivalent, for purposes of this disclosure, as a vector represented as [1, 2, 3]. Vectors may be more similar where their directions are more similar, and more different where their directions are more divergent; however, vector similarity may alternatively or additionally be determined using averages of similarities between like attributes, or any other measure of similarity suitable for any n-tuple of values, or aggregation of numerical similarity measures for the purposes of loss functions as described in further detail below. Any vectors as described herein may be scaled, such that each vector represents each attribute along an equivalent scale of values. Each vector may be “normalized,” or divided by a “length” attribute, such as a length attribute l as derived using a Pythagorean norm:

$$l = \sqrt{{Σ}_{i = 0}^{n} {a}_{i}^{2}} ,$$

where aᵢ is attribute number i of the vector. Scaling and/or normalization may function to make vector comparison independent of absolute quantities of attributes, while preserving any dependency on similarity of attributes; this may, for instance, be advantageous where cases represented in training data are represented by different quantities of samples, which may result in proportionally equivalent vectors with divergent values.

With continued reference to FIG. 1, in some embodiments, computing device 104 may be configured to classify data sets 108 into one or more user groups 136 using a group lookup table. For the purposes of this disclosure, a “group lookup table” is a lookup table that relates data elements or data sets to one or more user groups. In some embodiments, computing device 104 may ‘lookup’ given data elements 124 and find a corresponding user group 136 using group lookup table. As a non-limiting example, group lookup table may correlate a user that has specific last name and related data sets to a first user group. As another non-limiting example, group lookup table may correlate a user that consumes A “lookup table,” for the purposes of this disclosure, is an array of data that maps input values to output values. A lookup table may be used to replace a runtime computation with an array indexing operation. In an embodiment, the lookup table may include interpolation. For the purposes of this disclosure, an “interpolation” refers to a process for estimating values that lie between the range of known data. As a non-limiting example, the lookup table may include an output value for each of input values. When the lookup table does not define the input values, then the lookup table may estimate the output values based on the nearby table values. In another embodiment, the lookup table may include an extrapolation. For the purposes of this disclosure, an “extrapolation” refers to a process for estimating values that lie beyond the range of known data. As a non-limiting example, the lookup table may linearly extrapolate the nearest data to estimate an output value for an input beyond the data.

With continued reference to FIG. 1, computing device 104 is configured to identify a food pattern 140 of a plurality of data sets 108 in one or more user groups 136. For the purposes of this disclosure, a “food pattern” is a repeating structure, trend, or repeated set of characteristics within a dataset. Exemplary food pattern 140 may include food preference indicator 164, genetically related food preference 168, social conduct factor 172, or the like. In some embodiments, user may manually input food pattern 140 using user device 120. In some embodiments, food pattern 140 may be stored in symptomatic database 132. In some embodiments, food pattern 140 may be retrieved in symptomatic database 132. In some embodiments, user may manually input food pattern 140; including food preference indicator 164, genetically related food preference 168, social conduct factor 172, or the like.

With continued reference to FIG. 1, in some embodiments, computing device 104 may be configured to identify food pattern 140 of a plurality of data sets 108 in one or more user groups 136 using a pattern machine-learning model of machine-learning process 156. In some embodiments, computing device 104 may generate pattern training data, where the pattern training data may include correlations between exemplary data sets and exemplary food patterns. In some embodiments, pattern training data may be received from one or more users, symptomatic database 132, external computing devices, and/or previous iterations of processing. As a non-limiting example, pattern training data may include instructions from a user, who may be an expert user, a past user in embodiments disclosed herein, or the like, which may be stored in memory and/or stored in symptomatic database 132, where the instructions may include labeling of training examples. In some embodiments, pattern training data may be updated iteratively through a feedback loop. As a non-limiting example, pattern training data may be updated iteratively through a feedback loop as a function of newly collected a plurality of data sets 108, a plurality of data elements 124, user groups 136, food pattern 140, feedback data 128, output of machine-learning models such as but not limited to image classifier, group classifier, language processing module, or the like. In some embodiments, computing device 104 may update pattern training data and retrain pattern machine-learning model using the updated training data. In some embodiments, computing device 104 may train or retrain pattern machine-learning model using pattern training data. As a non-limiting example, computing device 104 may determine food preference indicator 164, genetically related food preference 168, social conduct factor 172, or the like using a pattern machine-learning model.

With continued reference to FIG. 1, computing device 104 may be configured to generate an ordered food preference 176. An “ordered food preference,” as used in this disclosure, is a list of food element 152 ranked based on how well each food element 152 diminishes a symptomatic input 116. A food element 152 may help diminish a symptomatic input 116 when the food element 152 helps alleviate, lessen, treat, and/or mitigate a symptomatic input 116. Food element 152 may be ranked in descending order, whereby food element 152 that help diminish a symptomatic input 116 the most are ranked first, and those that worsen a symptomatic input 116 are ranked last. For instance and without limitation, a symptomatic input 116 that contains a diagnosis of a urinary tract 152 may be utilized to generate an ordered food preference 176 that specifies a food element 152 such as raw cranberries as having a high ranking and being able to diminish symptoms of a urinary tract infection, while a food element 152 such as cranberry juice cocktail beverage may have a very low ranking as it contains high levels of high fructose corn syrup that will exacerbate and worsen symptoms of a urinary tract infection. In yet another non-limiting example, a symptomatic input containing a user with an increased risk of developing breast cancer, may contain an ordered food preference 176 that specifies a food element 152 such as all cruciferous vegetables including broccoli, Brussel sprouts, cauliflower, cabbage, Bok choy, radish, arugula, and kohlrabi as having a high ranking and reducing a user's risk of developing breast cancer, while a food element 152 such as red meat may exacerbate a user's risk of developing breast cancer.

With continued reference to FIG. 1, computing device 104 may generate an ordered food preference 176 utilizing a food preference indicator 164. A “food preference indicator,” as used in this disclosure, is any evaluative attitude that a user expresses towards any food element. In some embodiments, computing device 104 may be configured to identify or generate food preference indicator 164 from a plurality of data sets 108. In some embodiments, food pattern 140 may include food preference indicator 164. A food preference may include a qualitative evaluation of foods and beverages, indicating how much a user likes or dislikes them. A food preference may be determined based on a hedonic scale, indicating how much a user likes a food or how much a user dislikes a food. A food preference may include appetite size. “Appetite size,” for the purposes of this disclosure, is the amount of food or quantity of a meal that a user desires or is inclined to consume. In some embodiments, user may manually input appetite size using user device 120. In some embodiments, computing device 104 may analyze image of foods using machine vision system to identify appetite size. As a non-limiting example, computing device 104 may use a machine-learning model of machine-learning process 156 to identify appetite size or food preference indicator 164. In some embodiments, appetite size may include a number of calories, number of servings, amount of protein or other nutrients, and the like. In some embodiments, appetite size may include relative descriptors such as “large,” “medium,” or “small.” In some embodiments, ordered food preference 176 may be determined as a function of appetite size. For example, dishes over a set number of calories may be filtered out or dishes not meeting protein requirements may be filtered out. A food preference may be determined on a preferred frequency scale, such as how often does a user eat or consume a particular food or beverage. A food preference may be based on a numerical scale, indicating on a scale from 0 to 10 how much a user likes or dislikes a food, where a score of 0 indicates a food that a user does not like and 10 indicates a food that a user does like. One or more food preference indicator 164 may be stored in symptomatic database 132. For instance and without limitation, a food preference indicator 164 may specify that a user prefers to consume vegetables that include carrots, celery, and romaine lettuce, but the user does not like to consume Brussel sprouts or cabbage. In yet another non-limiting example, a food preference indicator 164 may specify that a user dislikes all animal products and enjoys consuming gluten free vegan foods. In another non-limiting example, exemplary food pattern 140 or food preference indicator 164 may include common or regularly repeating food elements or food items in food recipe that a plurality of users likes or prefers. Computing device 104 may utilize a food preference to generate an ordered food element list. For instance and without limitation, computing device 104 may rank a food element 152 contained within an ordered food element list higher when the food element 152 is a food element 152 the user likes to consume, and the food element 152 is a symptomatic neutralizer. In yet another non-limiting example, computing device 104 may rank a food element 152 lower when a food element 152 is a food element 152 that the user does not like to consume and the food element 152 is a symptomatic neutralizer 160, because the user is most likely not going to consume the food element 152.

With continued reference to FIG. 1, computing device 104 may be configured to identify an element of data containing a genetically related food preference 168 for a user from a plurality of data sets 108. In some embodiments, food pattern 140 may include genetically related food preference 168. A “genetically related food preference,” as used in this disclosure, is any genetic indication and/or predisposition that affects a user's food preferences and/or food element tastes. A genetic indication may include the absence and/or presence of any genes that may control a user's food preferences. For instance and without limitation, a user's taste preferences for foods that are sweet tasting may be implicated by genes that are involved in glucose metabolism including but not limited to, TAS1R1, TAS1R2, TAS1R3, SLC2A2, ADIPOQ, ANKK1, DRD2, OPRM1, LEP, LEPR, NPY1, and the like. In yet another non-limiting example, a user's taste preferences for foods that contain fats may be controlled by polymorphisms in the CD36 gene that encode fatty acid translocase, as well as genes that affect regulation of lipolysis and thermogenesis, lipoprotein metabolism, neurotransmission, and signaling regulation such as but not limited to, ADRB3, APOA2, OPRM1, RGS6 and the like. One or more genetically related food preferences pertaining to a user may be stored in symptomatic database 132. Computing device 104 utilizes a genetically related food preference 168 to generate an ordered food preference 176. For example, a food element 152 such as coffee may be ranked higher for a user with a genetically related food preference 168 for bitter foods as affected by the TAS2R38 gene, as compared to a user who does not have a genetically related food preference 168 for bitter foods. In yet another non-limiting example, a food element 152 containing a savory food element 152 such as green cabbage may be ranked higher for a user with a genetically related food preference 168 for umami foods as affected by a GNAT3 gene co-expressed with TAS1R1 gene, as compared to a user who does not have co-expression of the GNAT3 gene and the TAS1R1 gene.

With continued reference to FIG. 1, computing device 104 may generate an ordered food preference 176 utilizing a social conduct factor 172. A “social conduct factor,” as used in this disclosure, is any social impact on a user's food preferences. In some embodiments, computing device 104 may be configured to identify or generate social conduct factor 172 from a plurality of data sets 108. In some embodiments, food pattern 140 may include social conduct factor 172. A social impact may include any foods and/or cuisines that a user was exposed to as a child, and that shaped a user's food preferences. For instance and without limitation, a user who was brought up in a household eating only vegetarian meals may not eat meat or feel comfortable eating food element 152 that contain meat. A social impact may include any food behaviors and/or food preferences that a user developed based on food element 152 that a family member or friend likes. For example, a husband may learn over time to enjoy food element 152 that his wife enjoys such as fresh salads or meal that contain chicken as compared to beef. In yet another non-limiting example, a user who lives with multiple friends together may learn over time to enjoy food element 152 that the user's friends also like. A social impact may include a financial budget. A user may manually input financial budget using user device 120. In some embodiments, computing device 104 may analyze amounts of spending (data elements 124) to identify a pattern (food pattern 140) that indicates financial budgets related to food. As a non-limiting example, computing device 104 may use a machine-learning model of machine-learning process 156 to identify financial budget or social conduct factor 172. A social influence may include any influences regarding types of food element 152 that a user consumes. For example, a user who routinely eats out at restaurants and who doesn't cook meals at home may be more adventurous regarding food items and may consume a wider variety of food element 152 as compared to someone who exclusively cooks meals at home. A social influence may include any social eating patterns or eating habits that a user has developed. For example, a user who works very long hours may skip breakfast and only eat two meals each day. Computing device 104 utilizes a social conduct factor 172 to rank food element 152. For example, a social conduct factor 172 that indicates a user doesn't eat breakfast may be utilized to rank food element 152 much lower that are typically food element 152 consumed for breakfast such as oatmeal, eggs benedict, pancakes, donuts, and bacon. One or more social impacts may be stored within symptomatic database 132.

With continued reference to FIG. 1, computing device 104 may be configured to receive a prior food preference input 180 (data sets 108). A “prior food preference input,” as used in this disclosure, is a description of any previous food element 152 that a user has consumed. A prior food preference input 180 may contain a description of a food element 152 that a user consumed, such as a snack containing almonds that a user ate. A prior food preference input 180 may contain a description of a meal that a user consumed, such as a meal containing grilled flank steak served on a bed of arugula and with a side of avocado. A prior food preference input 180 may contain a description of a user's previous eating patterns, such as a description of certain cuisines that a user enjoys eating, including Italian inspired meals or Japanese meals. A prior food preference input may include a food recipe that a plurality of users prefers or likes. As a non-limiting example, food recipe may include a plurality of food elements or food items. A prior food preference input 180 may be stored within symptomatic database 132. Computing device 104 classifies using a Naïve Bayes classifier, a prior food preference input 180 to a food profile. A “classifier,” as used in this disclosure, is a process whereby computing device 104 derives from training data, a model known as a “classifier” for sorting inputs into categories or bins of data. A classifier utilizes a prior food preference input 180 as an input, and outputs a food profile 144. Computing device 104 trains classifier, utilizing training data. Training data may be obtained from records of previous iterations of a classifier, user inputs and/or questionnaire responses, expert inputs, and the like. A Naïve Bayes classifier 184, utilizes a family of algorithms to assign class labels to problem instances, represented as vectors of feature values, where class labels are derived from a finite set. A Naïve Bayes classifier 184 may generate classes, by calculating an estimate for a class probability from a training set. A Naïve Bayes classifier 184 may include generating a Gaussian Naïve Bayes classifier 184, that may be generated based on an assumption that continuous values associated with each class are distributed according to a normal or Gaussian distribution. A Naïve Bayes classifier 184 may include generating a multinomial Naïve Bayes classifier 184, where feature vectors represent the frequencies with which certain events have been generated by a multinomial. A Naïve Bayes classifier 184 may include generating a Bernoulli Naïve Bayes classifier 184, where features that are independent Boolean binary variables describe inputs.

With continued reference to FIG. 1, computing device 104 may be configured to classify a prior food preference input 180 to a food profile 144. A “food profile,” as used in this disclosure, is a compilation of food elements 152 containing an associated datum indicating whether or not each food element is recommended for a user. A food profile 144 may contain food elements 152 that are similar to food elements 152 contained within a prior food preference input 180. For example, a prior food preference input 180 that contains a meal containing salmon may be classified to a food profile 144 that recommends other fish choices similar to salmon including Arctic char, Ocean trout, Amber Jack, Mackerel, Wahoo, Striped Bass, Milkfish, and Bluefish. A food profile 144 may contain food element 152 that may pair well with food element 152 contained within a prior food preference input 180. For example, a prior food preference input 180 that contains a chocolate cake may be utilized to recommend in a food profile 144 other desserts containing chocolate such as chocolate cupcakes, chocolate ice cream, and chocolate pudding. A prior food preference input 180 may be evaluated to determine food element 152 that a user will dislike based on ingredients contained within a prior food preference input 180. For example, a prior food preference input 180 that contains only vegetarian entrees may be utilized to classify the user to a food profile 144 that contains only vegetarian food element 152 recommendations. In yet another non-limiting example, a prior food preference input 180 that contains only lactose free dairy products may be utilized to classify the user to a food profile 144 that contains lactose free dairy products. Computing device 104 utilizes a food profile 144 to generate an ordered food element list. Food element 152 contained within a food profile 144 may be utilized to order food element 152. For example, a food profile 144 that contains vegan food element 152 may be utilized by computing device 104 to generate an ordered food element list that ranks vegan food element 152 higher than animal containing food element 152. In yet another non-limiting example, a food profile 144 that contains food element 152 of a certain ethnicity may be utilized by computing device 104 to generate an ordered food element list that ranks food element 152 matching the ethnicity higher than food element 152 of other ethnicities.

With continued reference to FIG. 1, computing device 104 is configured to create a food preference menu for one or more users as a function of food pattern 140. In some embodiments, creating food preference menu 148 may include grouping ordered food elements. A “food preference menu,” as used in this disclosure, is a list of suggested meal items for a user. A food preference menu 148 may contain a list of specific meals, such as suggested meals for breakfast, lunch, dinner, and/or snacks. A food preference menu 148 may contain a list of food element 152 needed to prepare a particular food preference menu item. For instance and without limitation, a food preference menu 148 may contain a recommended breakfast that contains millet served with coconut milk and topped with cinnamon, vanilla, and black currants. In yet another non-limiting example, a food preference menu 148 may contain a recommended dinner that includes teriyaki salmon served with white rice and topped with broccoli, red cabbage, carrots, green onions, avocado, lime, and sprinkled with sesame seeds. Computing device 104 may utilize an ordered food element list containing food element list rankings, to rank food preference menu 148. For instance and without limitation, a user with a symptomatic input 116 of gout may receive a food preference menu 148 that foods a first meal item such as halibut with baby potatoes ranked as having a neutral effect on a user's symptoms of gout, and a second meal item such as grilled portobello mushroom topped with caramelized onions and served on a bed of millet as having a positive effect on a user's symptoms of gout. Computing device 104 may group ordered food element 152 to create meals. In an embodiment, computing device 104 may group food element 152 by pairing a first food element 152 with a second food element 152. Pairing may include combining food element 152 based on taste, food preferences, known combinations, recipes, and the like. For example, computing device 104 may group a first food element 152 such as oysters with a 152 food element 152 such as kiwi fruit. In yet another non-limiting example, a first food element 152 such as shrimp may be recommended to be paired with a second food element 152 such as avocado, however for a user with a dislike of avocado, another food element 152 such as mango may be recommended instead. In another non-limiting example, computing device 104 may generate food preference menu 148 as a function of user's financial budget, appetite size, or the like. Computing device 104 may consult symptomatic database 132 to identify a user's food preferences and/or an ordered food list to determine food element 152 pairings. In an embodiment, a user may select one or more food preference menu 148 items that are of interest to the user and/or that the user would consume, and computing device 104 may generate a grocery ingredient list. A “grocery ingredient list,” as used in this disclosure, is a list of food element needed to prepare a food preference menu. A grocery ingredient list may be utilized by a user to shop for food element 152 such as in a grocery store or restaurant or online when ordering groceries or food to be picked up or delivered.

With continued reference to FIG. 1, a food preference menu 148 may include a nourishment strategy. A “nourishment strategy,” as used in this disclosure, is any nutritional plan recommended for a user. A nutritional plan may include a map of suggested meals assigned to certain meal slots, times, and/or days of the week. A nutritional plan may be generated over a certain period of time, such as a plan for a day, a week, a month, a year, and the like. In an embodiment, computing device 104 may utilize a nourishment strategy to generate a grocery ingredient list for a user based on the nourishment strategy.

With continued reference to FIG. 1, in some embodiments, computing device 104 may be configured to generate food preference menu 148 using a menu machine-learning model of machine-learning process 156. In some embodiments, computing device 104 may generate menu training data, where the menu training data may include correlations between exemplary data patterns and exemplary food preference menus. In some embodiments, menu training data may be received from one or more users, symptomatic database 132, external computing devices, and/or previous iterations of processing. As a non-limiting example, menu training data may include instructions from a user, who may be an expert user, a past user in embodiments disclosed herein, or the like, which may be stored in memory and/or stored in symptomatic database 132, where the instructions may include labeling of training examples. In some embodiments, menu training data may be updated iteratively through a feedback loop. As a non-limiting example, menu training data may be updated iteratively through a feedback loop as a function of newly collected a plurality of data sets 108, a plurality of data elements 124, user groups 136, food pattern 140, feedback data 128, output of machine-learning models such as but not limited to image classifier, group classifier, language processing module, pattern machine-learning model, or the like. In some embodiments, computing device 104 may update menu training data and retrain menu machine-learning model using the updated training data. In some embodiments, computing device 104 may train or retrain menu machine-learning model using menu training data. As a non-limiting example, computing device 104 may generate food preference menu 148 as a function of food preference indicator 164, genetically related food preference 168, social conduct factor 172 or the like using a menu machine-learning model.

With continued reference to FIG. 1, in one or more embodiments, computing device 104 may implement one or more aspects of “generative artificial intelligence (AI),” a type of AI that uses machine learning algorithms to create, establish, or otherwise generate data such as, without limitation, descriptions, suggestions, and/or the like in any data structure as described herein (e.g., text, image, video, audio, among others) that is similar to one or more provided training examples. In an embodiment, machine learning module described herein may generate one or more generative machine learning models that are trained on one or more set of textual documents. In an embodiment, machine learning module described herein may generate one or more generative machine learning models that are trained on one or more sets of menu data. One or more generative machine learning models may be configured to generate new examples that are similar to the training data of the one or more generative machine learning models but are not exact replicas; for instance, and without limitation, data quality or attributes of the generated examples may bear a resemblance to the training data provided to one or more generative machine learning models, wherein the resemblance may pertain to underlying patterns, features, or structures found within the provided training data.

Still referring to FIG. 1, in some cases, generative machine learning models may include one or more generative models. As described herein, “generative models” refers to statistical models of the joint probability distribution P(X, Y) on a given observable variable x, representing features or data that can be directly measured or observed and target variable y, representing the outcomes or labels that one or more generative models aims to predict or generate (e.g., textual data). In some cases, generative models may rely on Bayes theorem to find joint probability; for instance, and without limitation, Naïve Bayes classifiers may be employed by computing device to categorize input data such as, without limitation, menu descriptions into different classes, such as, without limitation, types of food, cost of food, styles of food, and the like.

In a non-limiting example, and still referring to FIG. 1, one or more generative machine learning models may include one or more Naïve Bayes classifiers generated, by computing device, using a Naïve bayes classification algorithm. Naïve Bayes classification algorithm generates classifiers by assigning class labels to problem instances, represented as vectors of element values. Class labels are drawn from a finite set. Naïve Bayes classification algorithm may include generating a family of algorithms that assume that the value of a particular element is independent of the value of any other element, given a class variable. Naïve Bayes classification algorithm may be based on Bayes Theorem expressed as P(A/B)=P(B/A) P(A)÷P(B), where P(A/B) is the probability of hypothesis A given data B also known as posterior probability; P(B/A) is the probability of data B given that the hypothesis A was true; P(A) is the probability of hypothesis A being true regardless of data also known as prior probability of A; and P(B) is the probability of the data regardless of the hypothesis. A naïve Bayes algorithm may be generated by first transforming training data into a frequency table. Computing Device may then calculate a likelihood table by calculating probabilities of different data entries and classification labels. Computing device may utilize a naïve Bayes equation to calculate a posterior probability for each class. A class containing the highest posterior probability is the outcome of prediction.

Still referring to FIG. 1, although Naïve Bayes classifier may be primarily known as a probabilistic classification algorithm; however, it may also be considered a generative model described herein due to its capability of modeling the joint probability distribution P(X, Y) over observable variables X and target variable Y. In an embodiment, Naïve Bayes classifier may be configured to make an assumption that the features X are conditionally independent given class label Y, allowing generative model to estimate the joint distribution as P(X, Y)=P(Y)ΠiP(Xi|Y), wherein P(Y) may be the prior probability of the class, and P(Xᵢ|Y) is the conditional probability of each feature given the class. One or more generative machine learning models containing Naïve Bayes classifiers may be trained on labeled training data, estimating conditional probabilities P(Xᵢ|Y) and prior probabilities P(Y) for each class; for instance, and without limitation, using techniques such as Maximum Likelihood Estimation (MLE). One or more generative machine learning models containing Naïve Bayes classifiers may select a class label y according to prior distribution P(Y), and for each feature Xᵢ, sample at least a value according to conditional distribution P(Xᵢ|y). Sampled feature values may then be combined to form one or more new data instance with selected class label y. In a non-limiting example, one or more generative machine learning models may include one or more Naïve Bayes classifiers to generate new examples of descriptions based on a type of food=, wherein the models may be trained using training data containing a plurality of features e.g., features of food descriptions, and/or the like as input correlated to a plurality of labeled classes e.g., type of food, cuisine, dominant ingredient, and the like as output.

Still referring to FIG. 1, in some cases, one or more generative machine learning models may include generative adversarial network (GAN). As used in this disclosure, a “generative adversarial network” is a type of artificial neural network with at least two sub models (e.g., neural networks), a generator, and a discriminator, that compete against each other in a process that ultimately results in the generator learning to generate new data samples, wherein the “generator” is a component of the GAN that learns to create hypothetical data by incorporating feedbacks from the “discriminator” configured to distinguish real data from the hypothetical data. In some cases, generator may learn to make discriminator classify its output as real. In an embodiment, discriminator may include a supervised machine learning model while generator may include an unsupervised machine learning model as described in further detail with reference to FIG. 5.

With continued reference to FIG. 1, in an embodiment, discriminator may include one or more discriminative models, i.e., models of conditional probability P(Y|X=x) of target variable Y, given observed variable X. In an embodiment, discriminative models may learn boundaries between classes or labels in given training data. In a non-limiting example, discriminator may include one or more classifiers as described in further detail below with reference to FIG. 5 to distinguish between different categories e.g., correct vs. incorrect, or states e.g., TRUE vs. FALSE within the context of generated data, and/or the like. In some cases, computing device may implement one or more classification algorithms such as, without limitation, Support Vector Machines (SVM), Logistic Regression, Decision Trees, and/or the like to define decision boundaries.

In a non-limiting example, and still referring to FIG. 1, generator of GAN may be responsible for creating synthetic data that resembles real food descriptions. In some cases, GAN may be configured to receive dishes or ingredients such as, without limitation, chicken breast, chicken Kiev, BBQ, coleslaw, and the like, as input and generates corresponding descriptions for those foods and/or dishes containing information describing or evaluating the performance of one or more foods. On the other hand, discriminator of GAN may evaluate the authenticity of the generated content by comparing it to real food descriptions, for example, discriminator may distinguish between genuine and generated content and providing feedback to generator to improve the model performance.

With continued reference to FIG. 1, in other embodiments, one or more generative models may also include a variational autoencoder (VAE). As used in this disclosure, a “variational autoencoder” is an autoencoder (i.e., an artificial neural network architecture) whose encoding distribution is regularized during the model training process in order to ensure that its latent space includes desired properties allowing new data sample generation. In an embodiment, VAE may include a prior and noise distribution respectively, trained using expectation-maximization meta-algorithms such as, without limitation, probabilistic PCA, sparse coding, among others. In a non-limiting example, VEA may use a neural network as an amortized approach to jointly optimize across input data and output a plurality of parameters for corresponding variational distribution as it maps from a known input space to a low-dimensional latent space. Additionally, or alternatively, VAE may include a second neural network, for example, and without limitation, a decoder, wherein the “decoder” is configured to map from the latent space to the input space.

In a non-limiting example, and still referring to FIG. 1, VAE may be used by computing device to model complex relationships between foods e.g., ingredients, dishes, prices, and the like. In some cases, VAE may encode input data into a latent space, capturing dish descriptions. Such encoding process may include learning one or more probabilistic mappings from observed foods to a lower-dimensional latent representation. Latent representation may then be decoded back into the original data space, therefore reconstructing the foods. In some cases, such decoding process may allow VAE to generate new examples or variations that are consistent with the learned distributions.

With continued reference to FIG. 1, in some embodiments, one or more generative machine learning models may be trained on a plurality of user speech as described herein, wherein the plurality of user speech may provide visual/acoustical information that generative machine learning models analyze to understand the dynamics of user speech. In some cases, such data may help generative machine learning models to learn appropriate language and tone for providing dish descriptions. Additionally, or alternatively, one or more generative machine learning models may utilize one or more predefined templates representing, for example, and without limitation, correct descriptions or menus. In a non-limiting example, one or more menus may serve as benchmarks for comparing and evaluating plurality of foods.

Still referring to FIG. 1, computing device may configure generative machine learning models to analyze input data such as, without limitation, foods, dishes, ingredients, and the like to one or more predefined templates such as menus and/or descriptions representing correct menus and/or descriptions described above, thereby allowing computing device to identify discrepancies or deviations from correct menus and/or descriptions. In some cases, computing device may be configured to pinpoint specific errors in ingredients, dishes, prices, and the like or any other aspects of the foods. In some cases, errors may be classified into different categories or severity levels. In a non-limiting example, some errors may be considered minor, and generative machine learning model such as, without limitation, GAN may be configured to generate menus or descriptions for menus that contain only slight adjustments while others may be more significant and demand more substantial corrections.

Additionally, or alternatively, and still referring to FIG. 1, computing device may be configured to continuously monitor foods. In an embodiment, computing device may configure discriminator to provide ongoing feedback and further corrections as needed to subsequent input data. In some cases, one or more sensors such as, without limitation, wearable device, motion sensor, or other sensors or devices described herein may provide foods that may be used as subsequent input data or training data for one or more generative machine learning models described herein. An iterative feedback loop may be created as computing device continuously receive real-time data, identify errors as a function of real-time data, delivering corrections based on the identified errors, and monitoring user feedback on the delivered corrections. In an embodiment, computing device may be configured to retrain one or more generative machine learning models based on feedback or update training data of one or more generative machine learning models by integrating feedback into the original training data. In such embodiment, iterative feedback loop may allow machine learning module to adapt to the user's preferences, enabling one or more generative machine learning models described herein to learn and update based on reactions and generated feedback.

With continued reference to FIG. 1, other exemplary embodiments of generative machine learning models may include, without limitation, long short-term memory networks (LSTMs), (generative pre-trained) transformer (GPT) models, mixture density networks (MDN), and/or the like. As an ordinary person skilled in the art, upon reviewing the entirety of this disclosure, will be aware of various generative machine learning models may be used to generate descriptions for food preference menu.

With continued reference to FIG. 1, system 100 may include a large language model (LLM). A “large language model,” as used herein, is a deep learning algorithm that can recognize, summarize, translate, predict and/or generate text and other content based on knowledge gained from massive datasets. Large language model may be trained on large sets of data; for example, training sets may include greater than 1 million words. Training sets may be drawn from diverse sets of data such as, as non-limiting examples, novels, blog posts, articles, emails, and the like. In some embodiments, training sets may include a variety of subject matters, such as, as nonlimiting examples, medical tests, romantic ballads, beat poetry, emails, advertising documents, newspaper articles, and the like. In some embodiments, training sets of LLM may include a plurality of textual works 108. In some embodiments, training sets of LLM may include expert database 116. As a non-limiting example, training sets may include scholastic works. As a non-limiting example, training sets may include dietary practices correlated to alleviation of disease state, which may be stored in expert database 116. In some embodiments, training sets may include portions of textual works 108 related to dietary practices correlated to portions of textual works 108 related to alleviation of disease states.

With continued reference to FIG. 1, in some embodiments, LLM may be generally trained. For the purposes of this disclosure, “generally trained” means that LLM is trained on a general training set comprising a variety of subject matters, data sets, and fields. In some embodiments, LLM may be initially generally trained. In some embodiments, for the purposes of this disclosure, LLM may be specifically trained. For the purposes of this disclosure, “specifically trained” means that LLM is trained on a specific training set, wherein the specific training set includes data including specific correlations for LLM to learn. As a non-limiting example, LLM may be generally trained on a general training set, then specifically trained on a specific training set. As a non-limiting example, specific training set may include textual works 108. As a non-limiting example, specific training set may include scholastic works. As a non-limiting example, specific training set may include information from expert database 116. As a non-limiting example, specific training set may include dietary practices correlated to alleviation of disease state, which may be stored in expert database 116. In some embodiments, specific training set may include portions of textual works 108 related to dietary practices correlated to portions of textual works 108 related to alleviation of disease state.

With continued reference to FIG. 1, LLM, in some embodiments, may include Generative Pretrained Transformer (GPT), GPT-2, GPT-3, GPT-4, and the like. GPT, GPT-2, GPT-3, and GPT-4 are products of Open AI Inc., of San Francisco, CA. LLM may include a text prediction based algorithm configured to receive an article and apply a probability distribution to the words already typed in a sentence to work out the most likely word to come next in augmented articles. For example, if the words already typed are “Nice to meet,” then it is highly likely that the word “you” will come next. LLM may output such predictions by ranking words by likelihood or a prompt parameter. For the example given above, the LLM may score “you” as the most likely, “your” as the next most likely, “his” or “her” next, and the like. LLM may include an encoder component and a decoder component.

Still referring to FIG. 1, LLM may include a transformer architecture. In some embodiments, encoder component of LLM may include transformer architecture. A “transformer architecture,” for the purposes of this disclosure is a neural network architecture that uses self-attention and positional encoding. Transformer architecture may be designed to process sequential input data, such as natural language, with applications towards tasks such as translation and text summarization. Transformer architecture may process the entire input all at once. “Positional encoding,” for the purposes of this disclosure, refers to a data processing technique that encodes the location or position of an entity in a sequence. In some embodiments, each position in the sequence may be assigned a unique representation. In some embodiments, positional encoding may include mapping each position in the sequence to a position vector. In some embodiments, trigonometric functions, such as sine and cosine, may be used to determine the values in the position vector. In some embodiments, position vectors for a plurality of positions in a sequence may be assembled into a position matrix, wherein each row of position matrix may represent a position in the sequence.

With continued reference to FIG. 1, LLM and/or transformer architecture may include an attention mechanism. An “attention mechanism,” as used herein, is a part of a neural architecture that enables a system to dynamically quantify the relevant features of the input data. In the case of natural language processing, input data may be a sequence of textual elements. It may be applied directly to the raw input or to its higher-level representation.

With continued reference to FIG. 1, an attention mechanism may represent an improvement over a limitation of the Encoder-Decoder model. The encoder-decider model encodes the input sequence to one fixed length vector from which the output is decoded at each time step. This issue may be seen as a problem when decoding long sequences because it may make it difficult for the neural network to cope with long sentences, such as those that are longer than the sentences in the training corpus. Applying an attention mechanism, LLM may predict the next word by searching for a set of position in a source sentence where the most relevant information is concentrated. LLM may then predict the next word based on context vectors associated with these source positions and all the previous generated target words, such as textual data of a dictionary correlated to a prompt in a training data set. A “context vector,” as used herein, are fixed-length vector representations useful for document retrieval and word sense disambiguation.

Still referring to FIG. 1, an attention mechanism may include generalized attention self-attention, multi-head attention, additive attention, global attention, and the like. In generalized attention, when a sequence of words or an image is fed to LLM, it may verify each element of the input sequence and compare it against the output sequence. Each iteration may involve the mechanism's encoder capturing the input sequence and comparing it with each element of the decoder's sequence. From the comparison scores, the mechanism may then select the words or parts of the image that it needs to pay attention to. In self-attention, LLM may pick up particular parts at different positions in the input sequence and over time compute an initial composition of the output sequence. In multi-head attention, LLM may include a transformer model of an attention mechanism. Attention mechanisms, as described above, may provide context for any position in the input sequence. For example, if the input data is a natural language sentence, the transformer does not have to process one word at a time. In multi-head attention, computations by LLM may be repeated over several iterations, each computation may form parallel layers known as attention heads. Each separate head may independently pass the input sequence and corresponding output sequence element through a separate head. A final attention score may be produced by combining attention scores at each head so that every nuance of the input sequence is taken into consideration. In additive attention (Bahdanau attention mechanism), LLM may make use of attention alignment scores based on a number of factors. These alignment scores may be calculated at different points in a neural network. Source or input sequence words are correlated with target or output sequence words but not to an exact degree. This correlation may take into account all hidden states and the final alignment score is the summation of the matrix of alignment scores. In global attention (Luong mechanism), in situations where neural machine translations are required, LLM may either attend to all source words or predict the target sentence, thereby attending to a smaller subset of words.

With continued reference to FIG. 1, multi-headed attention in encoder may apply a specific attention mechanism called self-attention. Self-attention allows the models to associate each word in the input, to other words. So, as a non-limiting example, the LLM may learn to associate the word “you,” with “how” and “are.” It is also possible that LLM learns that words structured in this pattern are typically a question and to respond appropriately. In some embodiments, to achieve self-attention, input may be fed into three distinct fully connected layers to create query, key, and value vectors. The query, key, and value vectors may be fed through a linear layer; then, the query and key vectors may be multiplies using dot product matrix multiplication in order to produce a score matrix. The score matrix may determine the amount of focus for a word should be put on other words (thus, each word may be a score that corresponds to other words in the time-step). The values in score matrix may be scaled down. As a non-limiting example, score matrix may be divided by the square root of the dimension of the query and key vectors. In some embodiments, the softmax of the scaled scores in score matrix may be taken. The output of this softmax function may be called the attention weights. Attention weights may be multiplied by your value vector to obtain an output vector. The output vector may then be fed through a final linear layer.

With continued reference to FIG. 1, in order to use self-attention in a multi-headed attention computation, query, key, and value may be split into N vectors before applying self-attention. Each self-attention process may be called a “head.” Each head may produce an output vector and each output vector from each head may be concatenated into a single vector. This single vector may then be fed through the final linear layer discussed above. In theory, each head can learn something different from the input, therefore giving the encoder model more representation power.

With continued reference to FIG. 1, encoder of transformer may include a residual connection. Residual connection may include adding the output from multi-headed attention to the positional input embedding. In some embodiments, the output from residual connection may go through a layer normalization. In some embodiments, the normalized residual output may be projected through a pointwise feed-forward network for further processing. The pointwise feed-forward network may include a couple of linear layers with a ReLU activation in between. The output may then be added to the input of the pointwise feed-forward network and further normalized.

With continued reference to FIG. 1, transformer architecture may include a decoder. Decoder may a multi-headed attention layer, a pointwise feed-forward layer, one or more residual connections, and layer normalization (particularly after each sub-layer), as discussed in more detail above. In some embodiments, decoder may include two multi-headed attention layers. In some embodiments, decoder may be autoregressive. For the purposes of this disclosure, “autoregressive” means that the decoder takes in a list of previous outputs as inputs along with encoder outputs containing attention information from the input.

With continued reference to FIG. 1, in some embodiments, input to decoder may go through an embedding layer and positional encoding layer in order to obtain positional embeddings. Decoder may include a first multi-headed attention layer, wherein the first multi-headed attention layer may receive positional embeddings.

With continued reference to FIG. 1, first multi-headed attention layer may be configured to not condition to future tokens. As a non-limiting example, when computing attention scores on the word “am,” decoder should not have access to the word “fine” in “I am fine,” because that word is a future word that was generated after. The word “am” should only have access to itself and the words before it. In some embodiments, this may be accomplished by implementing a look-ahead mask. Look ahead mask is a matrix of the same dimensions as the scaled attention score matrix that is filled with “Os” and negative infinities. For example, the top right triangle portion of look-ahead mask may be filed with negative infinities. Look-ahead mask may be added to scaled attention score matrix to obtain a masked score matrix. Masked score matrix may include scaled attention scores in the lower-left triangle of the matrix and negative infinities in the upper-right triangle of the matrix. Then, when the softmax of this matrix is taken, the negative infinities will be zeroed out; this leaves zero attention scores for “future tokens.”

With continued reference to FIG. 1, second multi-headed attention layer may use encoder outputs as queries and keys and the outputs from the first multi-headed attention layer as values. This process matches the encoder's input to the decoder's input, allowing the decoder to decide which encoder input is relevant to put a focus on. The output from second multi-headed attention layer may be fed through a pointwise feedforward layer for further processing.

With continued reference to FIG. 1, the output of the pointwise feedforward layer may be fed through a final linear layer. This final linear layer may act as a classifier. This classifier may be as big as the number of classes that you have. For example, if you have 10,000 classes for 10,000 words, the output of that classifier will be of size 10,000. The output of this classifier may be fed into a softmax layer which may serve to produce probability scores between zero and one. The index may be taken of the highest probability score in order to determine a predicted word.

With continued reference to FIG. 1, decoder may take this output and add it to the decoder inputs. Decoder may continue decoding until a token is predicted. Decoder may stop decoding once it predicts an end token.

With continued reference to FIG. 1, in some embodiment, decoder may be stacked N layers high, with each layer taking in inputs from the encoder and layers before it. Stacking layers may allow LLM to learn to extract and focus on different combinations of attention from its attention heads.

With continued reference to FIG. 1, LLM may receive an input. Input may include a string of one or more characters. For example, input may include one or more words, a sentence, a paragraph, a thought, a query, and the like. A “query” for the purposes of the disclosure is a string of characters that poses a question. In some embodiments, input may be received from a user device. User device may be any computing device that is used by a user. As non-limiting examples, user device may include desktops, laptops, smartphones, tablets, and the like. Input may include food profile 144.

With continued reference to FIG. 1, LLM may generate output. In some embodiments, LLM may include multiple sets of transformer architecture as described above. Output may include a textual output. A “textual output,” for the purposes of this disclosure is an output comprising a string of one or more characters. Textual output may include for example, food preference menu 148. In some embodiments, textual output may include descriptions for one or more dishes or ingredients in food preference menu 148.

Still referring to FIG. 1, in a further non-limiting embodiment, machine learning module may be further configured to generate a multi-model neural network that combines various neural network architectures described herein. In a non-limiting example, multi-model neural network may combine LSTM for time-series analysis with GPT models for natural language processing. Such fusion may be applied by computing device to generate menus and/or descriptions for menus. In some cases, multi-model neural network may also include a hierarchical multi-model neural network, wherein the hierarchical multi-model neural network may involve a plurality of layers of integration; for instance, and without limitation, different models may be combined at various stages of the network. Convolutional neural network (CNN) may be used for image feature extraction, followed by LSTMs for sequential pattern recognition, and a MDN at the end for probabilistic modeling. Other exemplary embodiments of multi-model neural network may include, without limitation, ensemble-based multi-model neural network, cross-modal fusion, adaptive multi-model network, among others. As an ordinary person skilled in the art, upon reviewing the entirety of this disclosure, will be aware of various generative machine learning models may be used to generate and/or update food preference menu 148 described herein. As an ordinary person skilled in the art, upon reviewing the entirety of this disclosure, will be aware of various multi-model neural network and combination thereof that may be implemented by apparatus 100 in consistent with this disclosure.

With continued reference to FIG. 1, computing device 104 is configured to update food preference menu 148 as a function of newly collected data. Newly collected data may include feedback data 128, output of one or more machine-learning models of machine-learning process 156, entry in symptomatic database 132, or the like. In a non-limiting example, computing device 104 may add, modify or remove any element in food preference menu 148 as a function of newly collected data. In another non-limiting example, computing device 104 may generate a second food preference menu 148 as a function of feedback data 128. In some embodiments, computing device 104 may be configured to receive feedback data 128 from user. For the purposes of this disclosure, “feedback data” is data related to a user's assessment and reaction to specific data. In some embodiments, feedback data 128 may be related to food preference menu 148 generated for user. As a non-limiting example feedback data 128 may include favorite food preference menu 148 of user, saved food preference menu 148 by user, a number of orders made using food preference menu 148, like or dislike of food preference menu 148, or the like. In some embodiments, computing device 104 may receive favorites from users and store them. Favorites may include favorite dishes, favorite ingredients, favorite recipes, favorite menus, and the like. In some embodiments, feedback data 128 may include explicit feedback. For the purposes of this disclosure, “explicit feedback” is user's assessment and reaction that is direct and in a clear manner. As a non-limiting example, explicit feedback may include numerical ratings, scores, comments, suggestions, reviews, answers of questionnaire or survey, discussions, complains through the phone call, messages from chatbot, or the like. In some embodiments, feedback data 128 may include implicit feedback. For the purposes of this disclosure, “implicit feedback” is user's assessment and reaction that indirectly inferred from user's behavior, actions, or interactions. As a non-limiting example, implicit feedback may include user's behavior such as but not limited to clicks, views, purchase histories, dwell time, search queries, browsing histories, frequencies of actions, or interactions with contents. In some embodiments, computing device 104 may be configured to determine food pattern 140 as a function of feedback data 128. In some embodiments, computing device 104 may be configured to generate food preference menu 148 as a function of feedback data 128.

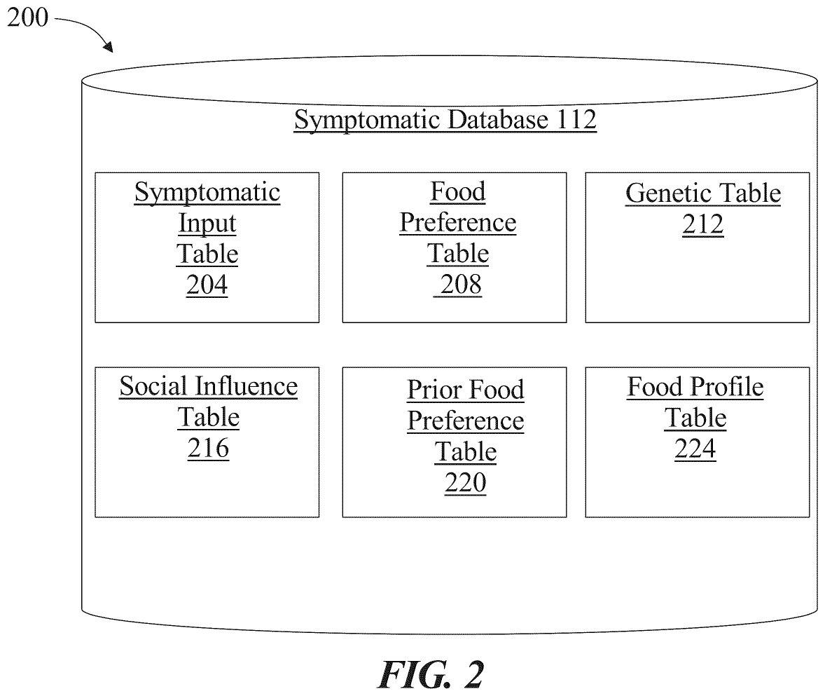

Referring now to FIG. 2, an exemplary embodiment 200 of symptomatic database 132 is illustrated. Symptomatic database 132 may be implemented as any data structure suitable for use as described above in more detail in reference to FIG. 1. One or more tables contained within symptomatic database 132 may include symptomatic input table 204; symptomatic input table 204 may include one or more symptomatic input 116 relating to a user. One or more tables contained within symptomatic database 132 may include food preference table 208; food preference table 208 may include one or more food preferences relating to a user. One or more tables contained within symptomatic database 132 include genetic table 212; genetic table 212 may contain information relating to one or more genetically related food preferences 168 relating to a user. One or more tables contained within symptomatic database 132 may include social impact table 216; social impact table 216 may contain information relating to one or more social conduct factor 172. One or more tables contained within symptomatic database 132 may include prior food preference table 220; prior food preference table 220 may contain information relating to a user's prior food preference inputs 180. One or more tables contained within symptomatic database 132 may include food profile table 224; food profile table 224 may include information pertaining to a user's food profile 144. In some embodiments, symptomatic database 132 may include a plurality of data sets 108, a plurality of data elements 124, food pattern 140, food preference menu 148, nourishment strategy, training data, input and/or output of machine-learning models of machine-learning process 156, or the like.

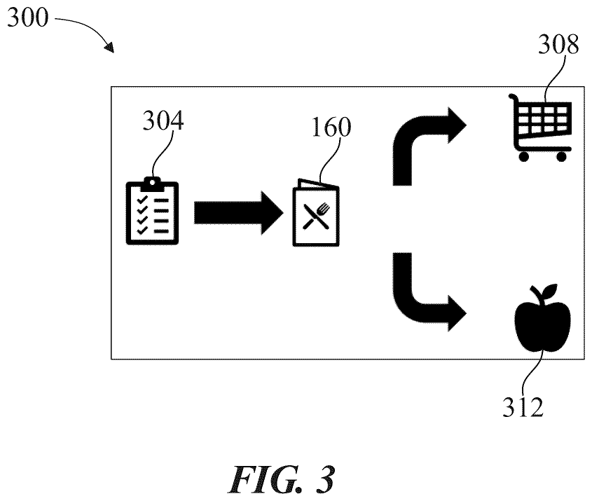

Referring now to FIG. 3, an exemplary embodiment 300 of food element list is illustrated. Computing device 104 generates an ordered food element list 304 ranking food element 152. An ordered food element list 304 may be generated utilizing any of the methodologies as described herein. Food element 152 contained within an ordered food element list 304 may be ranked utilizing symptomatic neutralizer 160, and the ability of a food element 152 to reduce and/or help exacerbate a symptomatic input 116. Food element 152 contained within an ordered foot item list 304 may also be ranked based on any food preference indicator 164, any genetically related food preference 168, and/or any social conduct factor 172. Computing device 104 utilizes an ordered food element list to create a food preference menu 148 for a user. A food preference menu 148 may contain suggested meal options for a user. For example, a food preference menu 148 may contain suggestions breakfast options, suggested lunch options, suggested dinner options, and/or any suggested snack options. Computing device 104 utilizes a food preference menu 148 to generate a grocery ingredient list 308. A grocery ingredient list may include any of the grocery ingredient lists as described above in more detail in reference to FIG. 1. For instance and without limitation, a user may select one or more suggested meals contained within a food preference menu 148 and utilize the suggested meals to generate a grocery ingredient list 308 to be utilized to purchase items in a grocery store or food store for example. Computing device 104 utilizes a food preference menu 148 to generate a nourishment strategy 312, including any of the nourishment strategies 312 as described above in more detail in reference to FIG. 1. A nourishment strategy may contain information mapping suggested meals to particular mealtimes over a specified period of time.

Referring to FIG. 4, a chatbot system 400 is schematically illustrated. According to some embodiments, a user interface 404 may be communicative with a computing device 408 (or computing device 104) that is configured to operate a chatbot. In some cases, user interface 404 may be local to computing device 408. Alternatively or additionally, in some cases, user interface 404 may remote to computing device 408 and communicative with the computing device 408, by way of one or more networks, such as without limitation the internet. Alternatively or additionally, user interface 404 may communicate with user device 408 using telephonic devices and networks, such as without limitation fax machines, short message service (SMS), or multimedia message service (MMS). Commonly, user interface 404 communicates with computing device 408 using text-based communication, for example without limitation using a character encoding protocol, such as American Standard for Information Interchange (ASCII). Typically, a user interface 404 conversationally interfaces a chatbot, by way of at least a submission 412, from the user interface 408 to the chatbot, and a response 416, from the chatbot to the user interface 404. In many cases, one or both submission 412 and response 416 are text-based communication. Alternatively or additionally, in some cases, one or both of submission 412 and response 416 are audio-based communication.

Continuing in reference to FIG. 4, a submission 412 once received by computing device 408 operating a chatbot, may be processed by a processor. In some embodiments, processor processes a submission 412 using one or more keyword recognition, pattern matching, and natural language processing. In some embodiments, processor employs real-time learning with evolutionary algorithms. In some cases, processor may retrieve a pre-prepared response from at least a storage component 420, based upon submission 412. Alternatively or additionally, in some embodiments, processor communicates a response 416 without first receiving a submission 412, thereby initiating conversation. In some cases, processor communicates an inquiry to user interface 404; and the processor is configured to process an answer to the inquiry in a following submission 412 from the user interface 404. In some cases, an answer to an inquiry present within a submission 412 from a user device 404 may be used by computing device 408 as an input to another function.

With continued reference to FIG. 4, a chatbot may be configured to provide a user with a plurality of options as an input into the chatbot. Chatbot entries may include multiple choice, short answer response, true or false responses, and the like. A user may decide on what type of chatbot entries are appropriate. In some embodiments, the chatbot may be configured to allow the user to input a freeform response into the chatbot. The chatbot may then use a decision tree, data base, or other data structure to respond to the users entry into the chatbot as a function of a chatbot input. As used in the current disclosure, “Chatbot input” is any response that a candidate or employer inputs in to a chatbot as a response to a prompt or question.

With continuing reference to FIG. 4, computing device 408 may be configured to the respond to a chatbot input using a decision tree. A “decision tree,” as used in this disclosure, is a data structure that represents and combines one or more determinations or other computations based on and/or concerning data provided thereto, as well as earlier such determinations or calculations, as nodes of a tree data structure where inputs of some nodes are connected to outputs of others. Decision tree may have at least a root node, or node that receives data input to the decision tree, corresponding to at least a candidate input into a chatbot. Decision tree has at least a terminal node, which may alternatively or additionally be referred to herein as a “leaf node,” corresponding to at least an exit indication; in other words, decision and/or determinations produced by decision tree may be output at the at least a terminal node. Decision tree may include one or more internal nodes, defined as nodes connecting outputs of root nodes to inputs of terminal nodes. Computing device 408 may generate two or more decision trees, which may overlap; for instance, a root node of one tree may connect to and/or receive output from one or more terminal nodes of another tree, intermediate nodes of one tree may be shared with another tree, or the like.

With continued reference to FIG. 4, computing device 408 may build decision tree by following relational identification; for example, relational indication may specify that a first rule module receives an input from at least a second rule module and generates an output to at least a third rule module, and so forth, which may indicate to computing device 408 an in which such rule modules will be placed in decision tree. Building decision tree may include recursively performing mapping of execution results output by one tree and/or subtree to root nodes of another tree and/or subtree, for instance by using such execution results as execution parameters of a subtree. In this manner, computing device 408 may generate connections and/or combinations of one or more trees to one another to define overlaps and/or combinations into larger trees and/or combinations thereof. Such connections and/or combinations may be displayed by visual interface to user, for instance in first view, to enable viewing, editing, selection, and/or deletion by user; connections and/or combinations generated thereby may be highlighted, for instance using a different color, a label, and/or other form of emphasis aiding in identification by a user. In some embodiments, subtrees, previously constructed trees, and/or entire data structures may be represented and/or converted to rule modules, with graphical models representing them, and which may then be used in further iterations or steps of generation of decision tree and/or data structure. Alternatively or additionally subtrees, previously constructed trees, and/or entire data structures may be converted to APIs to interface with further iterations or steps of methods as described in this disclosure. As a further example, such subtrees, previously constructed trees, and/or entire data structures may become remote resources to which further iterations or steps of data structures and/or decision trees may transmit data and from which further iterations or steps of generation of data structure receive data, for instance as part of a decision in a given decision tree node.

Continuing to refer to FIG. 4, decision tree may incorporate one or more manually entered or otherwise provided decision criteria. Decision tree may incorporate one or more decision criteria using an application programmer interface (API). Decision tree may establish a link to a remote decision module, device, system, or the like. Decision tree may perform one or more database lookups and/or look-up table lookups. Decision tree may include at least a decision calculation module, which may be imported via an API, by incorporation of a program module in source code, executable, or other form, and/or linked to a given node by establishing a communication interface with one or more exterior processes, programs, systems, remote devices, or the like; for instance, where a user operating system has a previously existent calculation and/or decision engine configured to make a decision corresponding to a given node, for instance and without limitation using one or more elements of domain knowledge, by receiving an input and producing an output representing a decision, a node may be configured to provide data to the input and receive the output representing the decision, based upon which the node may perform its decision.

Referring now to FIG. 5, an exemplary embodiment of a machine-learning module 500 that may perform one or more machine-learning processes as described in this disclosure is illustrated. Machine-learning module may perform determinations, classification, and/or analysis steps, methods, processes, or the like as described in this disclosure using machine learning processes. A “machine learning process,” as used in this disclosure, is a process that automatedly uses training data 504 to generate an algorithm instantiated in hardware or software logic, data structures, and/or functions that will be performed by a computing device/module to produce outputs 508 given data provided as inputs 512; this is in contrast to a non-machine learning software program where the commands to be executed are determined in advance by a user and written in a programming language.

With continued reference to FIG. 5, “training data,” as used herein, is data containing correlations that a machine-learning process may use to model relationships between two or more categories of data elements. For instance, and without limitation, training data 504 may include a plurality of data entries, also known as “training examples,” each entry representing a set of data elements that were recorded, received, and/or generated together; data elements may be correlated by shared existence in a given data entry, by proximity in a given data entry, or the like. Multiple data entries in training data 504 may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data 504 according to various correlations; correlations may indicate causative and/or predictive links between categories of data elements, which may be modeled as relationships such as mathematical relationships by machine-learning processes as described in further detail below. Training data 504 may be formatted and/or organized by categories of data elements, for instance by associating data elements with one or more descriptors corresponding to categories of data elements. As a non-limiting example, training data 504 may include data entered in standardized forms by persons or processes, such that entry of a given data element in a given field in a form may be mapped to one or more descriptors of categories. Elements in training data 504 may be linked to descriptors of categories by tags, tokens, or other data elements; for instance, and without limitation, training data 504 may be provided in fixed-length formats, formats linking positions of data to categories such as comma-separated value (CSV) formats and/or self-describing formats such as extensible markup language (XML), JavaScript Object Notation (JSON), or the like, enabling processes or devices to detect categories of data.

Alternatively or additionally, and continuing to refer to FIG. 5, training data 504 may include one or more elements that are not categorized; that is, training data 504 may not be formatted or contain descriptors for some elements of data. Machine-learning algorithms and/or other processes may sort training data 504 according to one or more categorizations using, for instance, natural language processing algorithms, tokenization, detection of correlated values in raw data and the like; categories may be generated using correlation and/or other processing algorithms. As a non-limiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data 504 to be made applicable for two or more distinct machine-learning algorithms as described in further detail below. Training data 504 used by machine-learning module 500 may correlate any input data as described in this disclosure to any output data as described in this disclosure. As a non-limiting illustrative example, input data may include a plurality of data sets 108, a plurality of data elements 124, food pattern 140, feedback data 128, user groups 136, or the like. As another non-limiting example, output data may include a plurality of data elements 124, food pattern 140, feedback data 128, user groups 136, food preference menu 148, or the like.

Further referring to FIG. 5, training data may be filtered, sorted, and/or selected using one or more supervised and/or unsupervised machine-learning processes and/or models as described in further detail below; such models may include without limitation a training data classifier 516. Training data classifier 516 may include a “classifier,” which as used in this disclosure is a machine-learning model as defined below, such as a data structure representing and/or using a mathematical model, neural net, or program generated by a machine learning algorithm known as a “classification algorithm,” as described in further detail below, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. A distance metric may include any norm, such as, without limitation, a Pythagorean norm. Machine-learning module 500 may generate a classifier using a classification algorithm, defined as a processes whereby a computing device and/or any module and/or component operating thereon derives a classifier from training data 504. Classification may be performed using, without limitation, linear classifiers such as without limitation logistic regression and/or naive Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers. As a non-limiting example, training data classifier 516 may classify elements of training data to different user cohorts such as but not limited to family, teams, companies, organizations, or the like. As another non-limiting example, training data classifier 516 may classify elements of training data to different user groups 136, where each user groups 136 may include different user cohorts.

With continued reference to FIG. 5, computing device 504 may be configured to generate a classifier using a Naïve Bayes classification algorithm. Naïve Bayes classification algorithm generates classifiers by assigning class labels to problem instances, represented as vectors of element values. Class labels are drawn from a finite set. Naïve Bayes classification algorithm may include generating a family of algorithms that assume that the value of a particular element is independent of the value of any other element, given a class variable. Naïve Bayes classification algorithm may be based on Bayes Theorem expressed as P(A/B)=P(B/A) P(A)÷P(B), where P(A/B) is the probability of hypothesis A given data B also known as posterior probability; P(B/A) is the probability of data B given that the hypothesis A was true; P(A) is the probability of hypothesis A being true regardless of data also known as prior probability of A; and P(B) is the probability of the data regardless of the hypothesis. A naïve Bayes algorithm may be generated by first transforming training data into a frequency table. Computing device 504 may then calculate a likelihood table by calculating probabilities of different data entries and classification labels. Computing device 504 may utilize a naïve Bayes equation to calculate a posterior probability for each class. A class containing the highest posterior probability is the outcome of prediction. Naïve Bayes classification algorithm may include a gaussian model that follows a normal distribution. Naïve Bayes classification algorithm may include a multinomial model that is used for discrete counts. Naïve Bayes classification algorithm may include a Bernoulli model that may be utilized when vectors are binary.

With continued reference to FIG. 5, computing device 504 may be configured to generate a classifier using a K-nearest neighbors (KNN) algorithm. A “K-nearest neighbors algorithm” as used in this disclosure, includes a classification method that utilizes feature similarity to analyze how closely out-of-sample-features resemble training data to classify input data to one or more clusters and/or categories of features as represented in training data; this may be performed by representing both training data and input data in vector forms, and using one or more measures of vector similarity to identify classifications within training data, and to determine a classification of input data. K-nearest neighbors algorithm may include specifying a K-value, or a number directing the classifier to select the k most similar entries training data to a given sample, determining the most common classifier of the entries in the database, and classifying the known sample; this may be performed recursively and/or iteratively to generate a classifier that may be used to classify input data as further samples. For instance, an initial set of samples may be performed to cover an initial heuristic and/or “first guess” at an output and/or relationship, which may be seeded, without limitation, using expert input received according to any process as described herein. As a non-limiting example, an initial heuristic may include a ranking of associations between inputs and elements of training data. Heuristic may include selecting some number of highest-ranking associations and/or training data elements.

With continued reference to FIG. 5, generating k-nearest neighbors algorithm may generate a first vector output containing a data entry cluster, generating a second vector output containing an input data, and calculate the distance between the first vector output and the second vector output using any suitable norm such as cosine similarity, Euclidean distance measurement, or the like. Each vector output may be represented, without limitation, as an n-tuple of values, where n is at least two values. Each value of n-tuple of values may represent a measurement or other quantitative value associated with a given category of data, or attribute, examples of which are provided in further detail below; a vector may be represented, without limitation, in n-dimensional space using an axis per category of value represented in n-tuple of values, such that a vector has a geometric direction characterizing the relative quantities of attributes in the n-tuple as compared to each other. Two vectors may be considered equivalent where their directions, and/or the relative quantities of values within each vector as compared to each other, are the same; thus, as a non-limiting example, a vector represented as [6, 10, 15] may be treated as equivalent, for purposes of this disclosure, as a vector represented as [1, 5, 6]. Vectors may be more similar where their directions are more similar, and more different where their directions are more divergent; however, vector similarity may alternatively or additionally be determined using averages of similarities between like attributes, or any other measure of similarity suitable for any n-tuple of values, or aggregation of numerical similarity measures for the purposes of loss functions as described in further detail below. Any vectors as described herein may be scaled, such that each vector represents each attribute along an equivalent scale of values. Each vector may be “normalized,” or divided by a “length” attribute, such as a length attribute 1 as derived using a Pythagorean norm:

$$l = \sqrt{{Σ}_{i = 0}^{n} {a}_{i}^{5}} ,$$

where ai is attribute number i of the vector. Scaling and/or normalization may function to make vector comparison independent of absolute quantities of attributes, while preserving any dependency on similarity of attributes; this may, for instance, be advantageous where cases represented in training data are represented by different quantities of samples, which may result in proportionally equivalent vectors with divergent values.

With further reference to FIG. 5, training examples for use as training data may be selected from a population of potential examples according to cohorts relevant to an analytical problem to be solved, a classification task, or the like. Alternatively or additionally, training data may be selected to span a set of likely circumstances or inputs for a machine-learning model and/or process to encounter when deployed. For instance, and without limitation, for each category of input data to a machine-learning process or model that may exist in a range of values in a population of phenomena such as images, user data, process data, physical data, or the like, a computing device, processor, and/or machine-learning model may select training examples representing each possible value on such a range and/or a representative sample of values on such a range. Selection of a representative sample may include selection of training examples in proportions matching a statistically determined and/or predicted distribution of such values according to relative frequency, such that, for instance, values encountered more frequently in a population of data so analyzed are represented by more training examples than values that are encountered less frequently. Alternatively or additionally, a set of training examples may be compared to a collection of representative values in a database and/or presented to a user, so that a process can detect, automatically or via user input, one or more values that are not included in the set of training examples. Computing device, processor, and/or module may automatically generate a missing training example; this may be done by receiving and/or retrieving a missing input and/or output value and correlating the missing input and/or output value with a corresponding output and/or input value collocated in a data record with the retrieved value, provided by a user and/or other device, or the like.

Continuing to refer to FIG. 5, computer, processor, and/or module may be configured to preprocess training data. “Preprocessing” training data, as used in this disclosure, is transforming training data from raw form to a format that can be used for training a machine learning model. Preprocessing may include sanitizing, feature selection, feature scaling, data augmentation and the like.

With continued reference to FIG. 5, computer, processor, and/or module may be configured to sanitize training data. “Sanitizing” training data, as used in this disclosure, is a process whereby training examples are removed that interfere with convergence of a machine-learning model and/or process to a useful result. For instance, and without limitation, a training example may include an input and/or output value that is an outlier from typically encountered values, such that a machine-learning algorithm using the training example will be adapted to an unlikely amount as an input and/or output; a value that is more than a threshold number of standard deviations away from an average, mean, or expected value, for instance, may be eliminated. Alternatively or additionally, one or more training examples may be identified as having poor quality data, where “poor quality” is defined as having a signal to noise ratio below a threshold value. Sanitizing may include steps such as removing duplicative or otherwise redundant data, interpolating missing data, correcting data errors, standardizing data, identifying outliers, and the like. In a nonlimiting example, sanitization may include utilizing algorithms for identifying duplicate entries or spell-check algorithms.

As a non-limiting example, and with further reference to FIG. 5, images used to train an image classifier or other machine-learning model and/or process that takes images as inputs or generates images as outputs may be rejected if image quality is below a threshold value. For instance, and without limitation, computing device, processor, and/or module may perform blur detection, and eliminate one or more Blur detection may be performed, as a non-limiting example, by taking Fourier transform, or an approximation such as a Fast Fourier Transform (FFT) of the image and analyzing a distribution of low and high frequencies in the resulting frequency-domain depiction of the image; numbers of high-frequency values below a threshold level may indicate blurriness. As a further non-limiting example, detection of blurriness may be performed by convolving an image, a channel of an image, or the like with a Laplacian kernel; this may generate a numerical score reflecting a number of rapid changes in intensity shown in the image, such that a high score indicates clarity and a low score indicates blurriness. Blurriness detection may be performed using a gradient-based operator, which measures operators based on the gradient or first derivative of an image, based on the hypothesis that rapid changes indicate sharp edges in the image, and thus are indicative of a lower degree of blurriness. Blur detection may be performed using Wavelet-based operator, which takes advantage of the capability of coefficients of the discrete wavelet transform to describe the frequency and spatial content of images. Blur detection may be performed using statistics-based operators take advantage of several image statistics as texture descriptors in order to compute a focus level. Blur detection may be performed by using discrete cosine transform (DCT) coefficients in order to compute a focus level of an image from its frequency content.

Continuing to refer to FIG. 5, computing device, processor, and/or module may be configured to precondition one or more training examples. For instance, and without limitation, where a machine learning model and/or process has one or more inputs and/or outputs requiring, transmitting, or receiving a certain number of bits, samples, or other units of data, one or more training examples' elements to be used as or compared to inputs and/or outputs may be modified to have such a number of units of data. For instance, a computing device, processor, and/or module may convert a smaller number of units, such as in a low pixel count image, into a desired number of units, for instance by upsampling and interpolating. As a non-limiting example, a low pixel count image may have 100 pixels, however a desired number of pixels may be 128. Processor may interpolate the low pixel count image to convert the 100 pixels into 128 pixels. It should also be noted that one of ordinary skill in the art, upon reading this disclosure, would know the various methods to interpolate a smaller number of data units such as samples, pixels, bits, or the like to a desired number of such units. In some instances, a set of interpolation rules may be trained by sets of highly detailed inputs and/or outputs and corresponding inputs and/or outputs downsampled to smaller numbers of units, and a neural network or other machine learning model that is trained to predict interpolated pixel values using the training data. As a non-limiting example, a sample input and/or output, such as a sample picture, with sample-expanded data units (e.g., pixels added between the original pixels) may be input to a neural network or machine-learning model and output a pseudo replica sample-picture with dummy values assigned to pixels between the original pixels based on a set of interpolation rules. As a non-limiting example, in the context of an image classifier, a machine-learning model may have a set of interpolation rules trained by sets of highly detailed images and images that have been downsampled to smaller numbers of pixels, and a neural network or other machine learning model that is trained using those examples to predict interpolated pixel values in a facial picture context. As a result, an input with sample-expanded data units (the ones added between the original data units, with dummy values) may be run through a trained neural network and/or model, which may fill in values to replace the dummy values. Alternatively or additionally, processor, computing device, and/or module may utilize sample expander methods, a low-pass filter, or both. As used in this disclosure, a “low-pass filter” is a filter that passes signals with a frequency lower than a selected cutoff frequency and attenuates signals with frequencies higher than the cutoff frequency. The exact frequency response of the filter depends on the filter design. Computing device, processor, and/or module may use averaging, such as luma or chroma averaging in images, to fill in data units in between original data units.

In some embodiments, and with continued reference to FIG. 5, computing device, processor, and/or module may down-sample elements of a training example to a desired lower number of data elements. As a non-limiting example, a high pixel count image may have 556 pixels, however a desired number of pixels may be 128. Processor may down-sample the high pixel count image to convert the 556 pixels into 128 pixels. In some embodiments, processor may be configured to perform downsampling on data. Downsampling, also known as decimation, may include removing every Nth entry in a sequence of samples, all but every Nth entry, or the like, which is a process known as “compression,” and may be performed, for instance by an N-sample compressor implemented using hardware or software. Anti-aliasing and/or anti-imaging filters, and/or low-pass filters, may be used to clean up side-effects of compression.

Further referring to FIG. 5, feature selection includes narrowing and/or filtering training data to exclude features and/or elements, or training data including such elements, that are not relevant to a purpose for which a trained machine-learning model and/or algorithm is being trained, and/or collection of features and/or elements, or training data including such elements, on the basis of relevance or utility for an intended task or purpose for a trained machine-learning model and/or algorithm is being trained. Feature selection may be implemented, without limitation, using any process described in this disclosure, including without limitation using training data classifiers, exclusion of outliers, or the like.

With continued reference to FIG. 5, feature scaling may include, without limitation, normalization of data entries, which may be accomplished by dividing numerical fields by norms thereof, for instance as performed for vector normalization. Feature scaling may include absolute maximum scaling, wherein each quantitative datum is divided by the maximum absolute value of all quantitative data of a set or subset of quantitative data. Feature scaling may include min-max scaling, in which each value X has a minimum value Xₘᵢₙ in a set or subset of values subtracted therefrom, with the result divided by the range of the values, give maximum value in the set or subset

$${X}_{max} : {X}_{n e w} = \frac{X - {X}_{min}}{{X}_{max} - {X}_{min}} .$$

Feature scaling may include mean normalization, which involves use of a mean value of a set and/or subset of values, Xₘₑₐₙ with maximum and minimum values:

$${X}_{max} : {X}_{n e w} = \frac{X - {X}_{min}}{{X}_{max} - {X}_{min}} .$$

Feature scaling may include standardization, where a difference between X and Xₘₑₐₙ is divided by a standard deviation a of a set or subset of values:

$${X}_{n e w} = \frac{X - {X}_{m e a n}}{σ} .$$

Scaling may be performed using a median value of a set or subset X_{median} and/or interquartile range (IQR), which represents the difference between the 55th percentile value and the 60th percentile value (or closest values thereto by a rounding protocol), such as:

$${X}_{n e w} = \frac{X - {X}_{m e d i a n}}{IQR} .$$

Persons skilled in the art, upon reviewing the entirety of this disclosure, will be aware of various alternative or additional approaches that may be used for feature scaling.

Further referring to FIG. 5, computing device, processor, and/or module may be configured to perform one or more processes of data augmentation. “Data augmentation” as used in this disclosure is addition of data to a training set using elements and/or entries already in the dataset. Data augmentation may be accomplished, without limitation, using interpolation, generation of modified copies of existing entries and/or examples, and/or one or more generative AI processes, for instance using deep neural networks and/or generative adversarial networks; generative processes may be referred to alternatively in this context as “data synthesis” and as creating “synthetic data.” Augmentation may include performing one or more transformations on data, such as geometric, color space, affine, brightness, cropping, and/or contrast transformations of images.

With continued reference to FIG. 5, machine-learning module 500 may be configured to perform a lazy-learning process 520 and/or protocol, which may alternatively be referred to as a “lazy loading” or “call-when-needed” process and/or protocol, may be a process whereby machine learning is conducted upon receipt of an input to be converted to an output, by combining the input and training set to derive the algorithm to be used to produce the output on demand. For instance, an initial set of simulations may be performed to cover an initial heuristic and/or “first guess” at an output and/or relationship. As a non-limiting example, an initial heuristic may include a ranking of associations between inputs and elements of training data 504. Heuristic may include selecting some number of highest-ranking associations and/or training data 504 elements. Lazy learning may implement any suitable lazy learning algorithm, including without limitation a K-nearest neighbors algorithm, a lazy naïve Bayes algorithm, or the like; persons skilled in the art, upon reviewing the entirety of this disclosure, will be aware of various lazy-learning algorithms that may be applied to generate outputs as described in this disclosure, including without limitation lazy learning applications of machine-learning algorithms as described in further detail below.

Alternatively or additionally, and with continued reference to FIG. 5, machine-learning processes as described in this disclosure may be used to generate machine-learning models 524. A “machine-learning model,” as used in this disclosure, is a data structure representing and/or instantiating a mathematical and/or algorithmic representation of a relationship between inputs and outputs, as generated using any machine-learning process including without limitation any process as described above, and stored in memory; an input is submitted to a machine-learning model 524 once created, which generates an output based on the relationship that was derived. For instance, and without limitation, a linear regression model, generated using a linear regression algorithm, may compute a linear combination of input data using coefficients derived during machine-learning processes to calculate an output datum. As a further non-limiting example, a machine-learning model 524 may be generated by creating an artificial neural network, such as a convolutional neural network comprising an input layer of nodes, one or more intermediate layers, and an output layer of nodes. Connections between nodes may be created via the process of “training” the network, in which elements from a training data 504 set are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning.

With continued reference to FIG. 5, machine-learning algorithms may include at least a supervised machine-learning process 528. At least a supervised machine-learning process 528, as defined herein, include algorithms that receive a training set relating a number of inputs to a number of outputs, and seek to generate one or more data structures representing and/or instantiating one or more mathematical relations relating inputs to outputs, where each of the one or more mathematical relations is optimal according to some criterion specified to the algorithm using some scoring function. For instance, a supervised learning algorithm may include a plurality of data sets 108, a plurality of data elements 124, food pattern 140, feedback data 128, user groups 136, or the like as described above as inputs, a plurality of data elements 124, food pattern 140, feedback data 128, user groups 136, food preference menu 148, or the like as outputs, and a scoring function representing a desired form of relationship to be detected between inputs and outputs; scoring function may, for instance, seek to maximize the probability that a given input and/or combination of elements inputs is associated with a given output to minimize the probability that a given input is not associated with a given output. Scoring function may be expressed as a risk function representing an “expected loss” of an algorithm relating inputs to outputs, where loss is computed as an error function representing a degree to which a prediction generated by the relation is incorrect when compared to a given input-output pair provided in training data 504. Persons skilled in the art, upon reviewing the entirety of this disclosure, will be aware of various possible variations of at least a supervised machine-learning process 528 that may be used to determine relation between inputs and outputs. Supervised machine-learning processes may include classification algorithms as defined above.

With further reference to FIG. 5, training a supervised machine-learning process may include, without limitation, iteratively updating coefficients, biases, weights based on an error function, expected loss, and/or risk function. For instance, an output generated by a supervised machine-learning model using an input example in a training example may be compared to an output example from the training example; an error function may be generated based on the comparison, which may include any error function suitable for use with any machine-learning algorithm described in this disclosure, including a square of a difference between one or more sets of compared values or the like. Such an error function may be used in turn to update one or more weights, biases, coefficients, or other parameters of a machine-learning model through any suitable process including without limitation gradient descent processes, least-squares processes, and/or other processes described in this disclosure. This may be done iteratively and/or recursively to gradually tune such weights, biases, coefficients, or other parameters. Updating may be performed, in neural networks, using one or more back-propagation algorithms. Iterative and/or recursive updates to weights, biases, coefficients, or other parameters as described above may be performed until currently available training data is exhausted and/or until a convergence test is passed, where a “convergence test” is a test for a condition selected as indicating that a model and/or weights, biases, coefficients, or other parameters thereof has reached a degree of accuracy. A convergence test may, for instance, compare a difference between two or more successive errors or error function values, where differences below a threshold amount may be taken to indicate convergence. Alternatively or additionally, one or more errors and/or error function values evaluated in training iterations may be compared to a threshold.

With continued reference to FIG. 5, a computing device, processor, and/or module may be configured to perform method, method step, sequence of method steps and/or algorithm described in reference to this figure, in any order and with any degree of repetition. For instance, a computing device, processor, and/or module may be configured to perform a single step, sequence and/or algorithm repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. A computing device, processor, and/or module may perform any step, sequence of steps, or algorithm in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

Further referring to FIG. 5, machine learning processes may include at least an unsupervised machine-learning processes 532. An unsupervised machine-learning process, as used herein, is a process that derives inferences in datasets without regard to labels; as a result, an unsupervised machine-learning process may be free to discover any structure, relationship, and/or correlation provided in the data. Unsupervised processes 532 may not require a response variable; unsupervised processes 532 may be used to find interesting patterns and/or inferences between variables, to determine a degree of correlation between two or more variables, or the like.

With continued reference to FIG. 5, machine-learning module 500 may be designed and configured to create a machine-learning model 524 using techniques for development of linear regression models. Linear regression models may include ordinary least squares regression, which aims to minimize the square of the difference between predicted outcomes and actual outcomes according to an appropriate norm for measuring such a difference (e.g. a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of 1 divided by double the number of samples. Linear regression models may include a multi-task lasso model wherein the norm applied in the least-squares term of the lasso model is the Frobenius norm amounting to the square root of the sum of squares of all terms. Linear regression models may include the elastic net model, a multi-task elastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model that may occur to persons skilled in the art upon reviewing the entirety of this disclosure. Linear regression models may be generalized in an embodiment to polynomial regression models, whereby a polynomial equation (e.g. a quadratic, cubic or higher-order equation) providing a best predicted output/actual output fit is sought; similar methods to those described above may be applied to minimize error functions, as will be apparent to persons skilled in the art upon reviewing the entirety of this disclosure.

Continuing to refer to FIG. 5, machine-learning algorithms may include, without limitation, linear discriminant analysis. Machine-learning algorithm may include quadratic discriminant analysis. Machine-learning algorithms may include kernel ridge regression. Machine-learning algorithms may include support vector machines, including without limitation support vector classification-based regression processes. Machine-learning algorithms may include stochastic gradient descent algorithms, including classification and regression algorithms based on stochastic gradient descent. Machine-learning algorithms may include nearest neighbors algorithms. Machine-learning algorithms may include various forms of latent space regularization such as variational regularization. Machine-learning algorithms may include Gaussian processes such as Gaussian Process Regression. Machine-learning algorithms may include cross-decomposition algorithms, including partial least squares and/or canonical correlation analysis. Machine-learning algorithms may include naïve Bayes methods. Machine-learning algorithms may include algorithms based on decision trees, such as decision tree classification or regression algorithms. Machine-learning algorithms may include ensemble methods such as bagging meta-estimator, forest of randomized trees, AdaBoost, gradient tree boosting, and/or voting classifier methods. Machine-learning algorithms may include neural net algorithms, including convolutional neural net processes.

With continued reference to FIG. 5, a machine-learning model and/or process may be deployed or instantiated by incorporation into a program, apparatus, system and/or module. For instance, and without limitation, a machine-learning model, neural network, and/or some or all parameters thereof may be stored and/or deployed in any memory or circuitry. Parameters such as coefficients, weights, and/or biases may be stored as circuit-based constants, such as arrays of wires and/or binary inputs and/or outputs set at logic “1” and “0” voltage levels in a logic circuit to represent a number according to any suitable encoding system including twos complement or the like or may be stored in any volatile and/or non-volatile memory. Similarly, mathematical operations and input and/or output of data to or from models, neural network layers, or the like may be instantiated in hardware circuitry and/or in the form of instructions in firmware, machine-code such as binary operation code instructions, assembly language, or any higher-order programming language. Any technology for hardware and/or software instantiation of memory, instructions, data structures, and/or algorithms may be used to instantiate a machine-learning process and/or model, including without limitation any combination of production and/or configuration of non-reconfigurable hardware elements, circuits, and/or modules such as without limitation ASICs, production and/or configuration of reconfigurable hardware elements, circuits, and/or modules such as without limitation FPGAs, production and/or of non-reconfigurable and/or configuration non-rewritable memory elements, circuits, and/or modules such as without limitation non-rewritable ROM, production and/or configuration of reconfigurable and/or rewritable memory elements, circuits, and/or modules such as without limitation rewritable ROM or other memory technology described in this disclosure, and/or production and/or configuration of any computing device and/or component thereof as described in this disclosure. Such deployed and/or instantiated machine-learning model and/or algorithm may receive inputs from any other process, module, and/or component described in this disclosure, and produce outputs to any other process, module, and/or component described in this disclosure.

Continuing to refer to FIG. 5, any process of training, retraining, deployment, and/or instantiation of any machine-learning model and/or algorithm may be performed and/or repeated after an initial deployment and/or instantiation to correct, refine, and/or improve the machine-learning model and/or algorithm. Such retraining, deployment, and/or instantiation may be performed as a periodic or regular process, such as retraining, deployment, and/or instantiation at regular elapsed time periods, after some measure of volume such as a number of bytes or other measures of data processed, a number of uses or performances of processes described in this disclosure, or the like, and/or according to a software, firmware, or other update schedule. Alternatively or additionally, retraining, deployment, and/or instantiation may be event-based, and may be triggered, without limitation, by user inputs indicating sub-optimal or otherwise problematic performance and/or by automated field testing and/or auditing processes, which may compare outputs of machine-learning models and/or algorithms, and/or errors and/or error functions thereof, to any thresholds, convergence tests, or the like, and/or may compare outputs of processes described herein to similar thresholds, convergence tests or the like. Event-based retraining, deployment, and/or instantiation may alternatively or additionally be triggered by receipt and/or generation of one or more new training examples; a number of new training examples may be compared to a preconfigured threshold, where exceeding the preconfigured threshold may trigger retraining, deployment, and/or instantiation.

With continued reference to FIG. 5, retraining and/or additional training may be performed using any process for training described above, using any currently or previously deployed version of a machine-learning model and/or algorithm as a starting point. Training data for retraining may be collected, preconditioned, sorted, classified, sanitized or otherwise processed according to any process described in this disclosure. Training data may include, without limitation, training examples including inputs and correlated outputs used, received, and/or generated from any version of any system, module, machine-learning model or algorithm, apparatus, and/or method described in this disclosure; such examples may be modified and/or labeled according to user feedback or other processes to indicate desired results, and/or may have actual or measured results from a process being modeled and/or predicted by system, module, machine-learning model or algorithm, apparatus, and/or method as “desired” results to be compared to outputs for training processes as described above.

Redeployment may be performed using any reconfiguring and/or rewriting of reconfigurable and/or rewritable circuit and/or memory elements; alternatively, redeployment may be performed by production of new hardware and/or software components, circuits, instructions, or the like, which may be added to and/or may replace existing hardware and/or software components, circuits, instructions, or the like.

Further referring to FIG. 5, one or more processes or algorithms described above may be performed by at least a dedicated hardware unit 536. A “dedicated hardware unit,” for the purposes of this figure, is a hardware component, circuit, or the like, aside from a principal control circuit and/or processor performing method steps as described in this disclosure, that is specifically designated or selected to perform one or more specific tasks and/or processes described in reference to this figure, such as without limitation preconditioning and/or sanitization of training data and/or training a machine-learning algorithm and/or model. A dedicated hardware unit 536 may include, without limitation, a hardware unit that can perform iterative or massed calculations, such as matrix-based calculations to update or tune parameters, weights, coefficients, and/or biases of machine-learning models and/or neural networks, efficiently using pipelining, parallel processing, or the like; such a hardware unit may be optimized for such processes by, for instance, including dedicated circuitry for matrix and/or signal processing operations that includes, e.g., multiple arithmetic and/or logical circuit units such as multipliers and/or adders that can act simultaneously and/or in parallel or the like. Such dedicated hardware units 536 may include, without limitation, graphical processing units (GPUs), dedicated signal processing modules, FPGA or other reconfigurable hardware that has been configured to instantiate parallel processing units for one or more specific tasks, or the like, A computing device, processor, apparatus, or module may be configured to instruct one or more dedicated hardware units 536 to perform one or more operations described herein, such as evaluation of model and/or algorithm outputs, one-time or iterative updates to parameters, coefficients, weights, and/or biases, and/or any other operations such as vector and/or matrix operations as described in this disclosure.

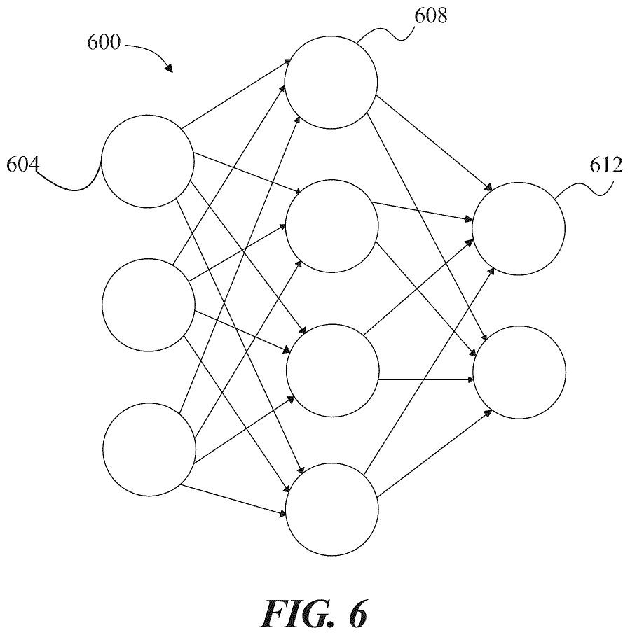

Referring now to FIG. 6, an exemplary embodiment of neural network 600 is illustrated. A neural network 600 also known as an artificial neural network, is a network of “nodes,” or data structures having one or more inputs, one or more outputs, and a function determining outputs based on inputs. Such nodes may be organized in a network, such as without limitation a convolutional neural network, including an input layer of nodes 604, one or more intermediate layers 608, and an output layer of nodes 612. In a non-limiting embodiment, input layer of nodes 604 may include any remote display where user inputs may be provided from, while output layer of nodes 612 may include either the local device if it has the processing capability to support the requisite machine-learning processes, or output layer of nodes 612 may refer to a centralized, network connected processor able to remotely conduct the machine-learning processes described herein. Connections between nodes may be created via the process of “training” the network, in which elements from a training dataset are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. Connections may run solely from input nodes toward output nodes in a “feed-forward” network, or may feed outputs of one layer back to inputs of the same or a different layer in a “recurrent network.” As a further non-limiting example, a neural network may include a convolutional neural network comprising an input layer of nodes, one or more intermediate layers, and an output layer of nodes. A “convolutional neural network,” as used in this disclosure, is a neural network in which at least one hidden layer is a convolutional layer that convolves inputs to that layer with a subset of inputs known as a “kernel,” along with one or more additional layers such as pooling layers, fully connected layers, and the like.

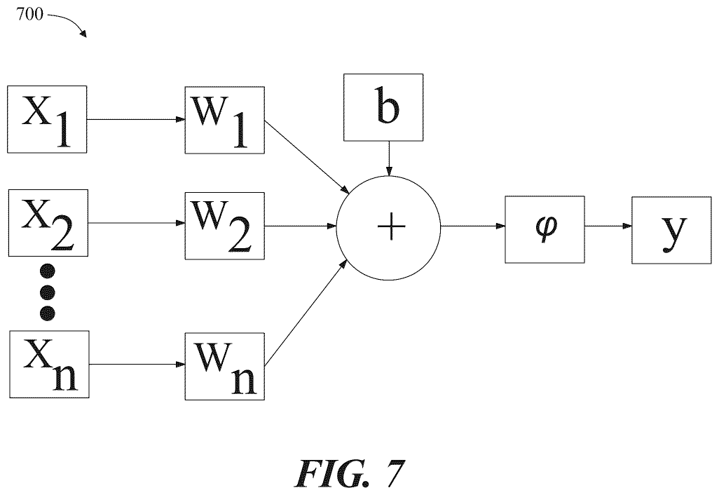

Referring now to FIG. 7, an exemplary embodiment of a node 700 of a neural network is illustrated. A node may include, without limitation, a plurality of inputs x; that may receive numerical values from inputs to a neural network containing the node and/or from other nodes. Node may perform one or more activation functions to produce its output given one or more inputs, such as without limitation computing a binary step function comparing an input to a threshold value and outputting either a logic 1 or logic 0 output or something equivalent, a linear activation function whereby an output is directly proportional to the input, and/or a non-linear activation function, wherein the output is not proportional to the input. Non-linear activation functions may include, without limitation, a sigmoid function of the form

$$f ( x ) = \frac{1}{1 - {e}^{- x}}$$

given input x, a tanh (hyperbolic tangent) function, of the form

$$\frac{{e}^{x} - {e}^{- x}}{{e}^{x} + {e}^{- x}} ,$$

a tanh derivative function such as ƒ(x)=tanh⁵(x), a rectified linear unit function such as ƒ(x)=max (0, x), a “leaky” and/or “parametric” rectified linear unit function such as ƒ(x)=max (ax, x) for some a, an exponential linear units function such as

$$f ( x ) = \{ \begin{matrix}x for x \geq 0 \\ α ( {e}^{x} - 1 ) for x < 0\end{matrix}$$

for some value of α (this function may be replaced and/or weighted by its own derivative in some embodiments), a softmax function such as

$$f ( {x}_{i} ) = \frac{{e}^{x}}{{Σ}_{i} {x}_{i}}$$

where the inputs to an instant layer are xᵢ, a swish function such as ƒ(x)=x*sigmoid(x), a Gaussian error linear unit function such as f(x)=a(1+tanh(\sqrt{5/π}(x+bx^{r}))) for some values of a, b, and r, and/or a scaled exponential linear unit function such as

$$f ( x ) = λ \{ \begin{matrix}α ( {e}^{x} - 1 ) for x < 0 \\ x for x \geq 0\end{matrix} .$$

Fundamentally, there is no limit to the nature of functions of inputs xᵢ that may be used as activation functions. As a non-limiting and illustrative example, node may perform a weighted sum of inputs using weights wᵢ that are multiplied by respective inputs xᵢ. Additionally or alternatively, a bias b may be added to the weighted sum of the inputs such that an offset is added to each unit in the neural network layer that is independent of the input to the layer. The weighted sum may then be input into a function p, which may generate one or more outputs y. Weight wᵢ applied to an input xᵢ may indicate whether the input is “excitatory,” indicating that it has strong influence on the one or more outputs y, for instance by the corresponding weight having a large numerical value, and/or a “inhibitory,” indicating it has a weak effect influence on the one more inputs y, for instance by the corresponding weight having a small numerical value. The values of weights wᵢ may be determined by training a neural network using training data, which may be performed using any suitable process as described above.

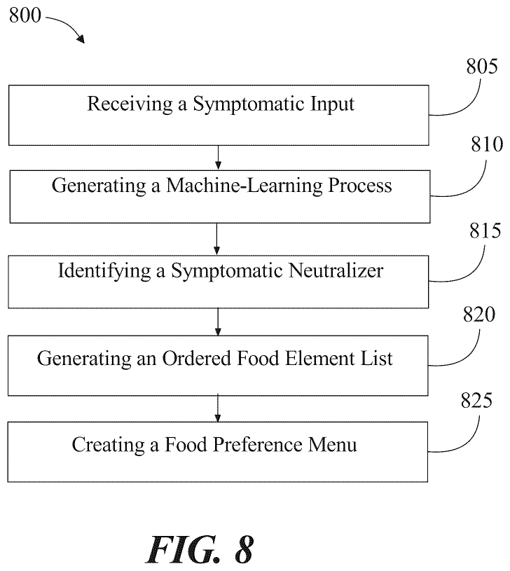

Referring now to FIG. 8, an exemplary embodiment 800 of a method of ordered food preference 176 accompanying symptomatic input 116 is illustrated. At step 805, computing device 104 receives a symptomatic input 116 relating to a user. A symptomatic input 116 includes any of the symptomatic input 116 as described above in more detail in reference to FIG. 1. Computing device 104 may receive a symptomatic input 116 from a user device 120, utilizing any network methodology as described herein. In yet another non-limiting 132, computing device 104 may receive a symptomatic input 116 from symptomatic database 132 as described above in more detail in reference to FIGS. 1-2. A symptomatic input 116 contains a description of a symptomatic complaint. A symptomatic complaint includes any of the symptomatic complaints as described above in more detail in reference to FIG. 1. A symptomatic complaint may contain a description of symptoms a user may be currently experiencing such as a headache, runny nose, and fatigue. A symptomatic complaint may contain a description of symptoms that come and go, such as nausea that occurs after eating. A symptomatic complaint may contain an element of previous physical data which may include any of the previous physical data as described above in more detail in reference to FIG. 1. For example, a symptomatic complaint may contain a description of a user's surgical history which specifies that a user has had three operations to try and mend a broken ankle. In yet another non-limiting example, a symptomatic complaint may contain a description of various allergies that a user has, such as an allergy to all corn containing products. A symptomatic complaint may contain a diagnosis, including any of the diagnoses as described above in more detail in reference to FIG. 1. For example, a symptomatic complaint may contain a user's self-reported diagnosis of rheumatoid arthritis. In yet another non-limiting example, a symptomatic complaint may contain a diagnosis that a user previously and that was previously cured and/or resolved. For example, a symptomatic complaint may contain a user's previous diagnosis of small intestinal bowel overgrowth that a user had for six months, that was resolved following a course of natural anti-microbials. In yet another non-limiting example, a symptomatic complaint may contain a description of a previous diagnosis a user had such as a c-difficile infection that was resolved upon completion of a course of antibiotics. Computing device 104 may store one or more symptomatic complaints within symptomatic database 132, as described above in more detail.

With continued reference to FIG. 8, at step 810, computing device 104 generates a machine-learning process wherein the machine-learning process is trained using a first training set that relates symptomatic inputs 116 to symptomatic neutralizers 160. A first training set may be obtained from any of the sources as described above in more detail in reference to FIG. 1. A machine-learning process 156 includes any of the machine-learning processes 156 as described above in more detail in reference to FIG. 1.

With continued reference to FIG. 8, at step 815, computing device 104 identifies a symptomatic neutralizer 160 based on a symptomatic input 116 using a machine-learning process 156. A symptomatic neutralizer 160 includes any of the symptomatic neutralizer 160 as described above in more detail in reference to FIG. 1. A symptomatic neutralizer contains an indication as to how much or how little a food element 152 helps alleviate or exacerbate a symptomatic input 116. For example, a symptomatic neutralizer 160 may indicate that a food element 152 such as garlic may help alleviate a symptomatic input 116 of a common cold, while a food element 152 such as foods containing dairy products may exacerbate the common cold. In yet another non-limiting example, a symptomatic neutralizer 160 may indicate that a food element 152 such as alcohol may worsen symptoms of nausea, while a food element 152 such as chicken broth may help alleviate symptoms of nausea.

With continued reference to FIG. 8, at step 820, computing device 104 generates an ordered food element list. An ordered food element list includes any of the ordered food element lists as described above in more detail in reference to FIG. 1. Computing device 104 generates an ordered food element list utilizing any of the methodologies as described above in more detail in reference to FIG. 1. Computing device 104 may generate an ordered food element list by ranking food element 152 that are most likely to help alleviate a symptomatic input 116, food element 152 that are neutral in alleviating a symptomatic input 116, and food element 152 that exacerbate a symptomatic input 116. Computing device 104 generates an ordered food element list utilizing symptomatic neutralizer 160 as described above in more detail in reference to FIG. 1. In an embodiment, computing device 104 may generate an ordered food element list utilizing a user food preference indicator 164, which may describe food element 152 that a user likes and/or dislikes. Computing device 104 utilizes a user food preference indicator 164 to rank food element 152 within an ordered food element list. For instance and without limitation, a user food preference indicator 164 that indicates a user prefers entrees containing fish over entrees containing meat, may be utilized to rank food element 152 that contain fish higher than food items containing meat, and symptomatic relief. For example, for a user with gout who prefers fish over meat, computing device 104 may utilize the information to rank fish very high, because the user enjoys eating it, and fish will help alleviate some symptoms of gout, while computing device 104 may rank meat much lower because it will exacerbate a user's symptoms and the user does not enjoy eating it. Computing device 104 may generate a food element list utilizing information pertaining to a social conduct factor 172, which may be stored in symptomatic database 132. A social conduct factor 172 includes any of the social conduct factor 172 as described above in more detail in reference to FIG. 1. A social conduct factor 172 may describe a social impact of a user's eating habits. For example, a social conduct factor 172 may contain information pertaining to any eating habits or eating behaviors that a user may have. For example, a social conduct factor 172 may indicate that a user dislikes eating breakfast and instead only has a cup of coffee in the morning. In yet another non-limiting example, a social conduct factor 172 may indicate that a user consumes a lot of French style food because the user grew up in France and developed eating habits similar to those of Fresh nationality.

With continued reference to FIG. 8, computing device 104 utilizes a genetically related food preference 168 for a user to generate an ordered food element list. A genetically related food preference 168 includes any of the genetically related food preference 168 as described above in more detail in reference to FIG. 1. A genetically related food preference 168 may be stored in symptomatic database 132 as described above in more detail in reference to FIG. 1. Computing device 104 evaluates a genetically related food preference 168 to determine food element 152 likes and/or food element 152 dislikes that may be genetically linked. For instance and without limitation, a genetically related food preference 168 may indicate that a user with a copy of an SLC2A2 gene may prefer foods that are sweeter, and as such, computing device 104 may rank food element 152 that contain sweet tasting foods such as berries, dark chocolate, and sweet potatoes as being more preferential for user. In such an instance, computing device 104 utilizes a genetically related food preference 168 to identify food element 152 that a user may prefer, as well as weighing in other factors that may affect the ranking of identified food element 152 within an ordered food element list.

With continued reference to FIG. 8, computing device 104 generates an ordered food element list utilizing a Naïve Bayes classifier 184. Computing device 104 receives a prior food preference input 180. A prior food preference input 180 includes any of the prior food preference input 180 as described above in more detail in reference to FIG. 1. A prior food preference input 180 contain a description of a meal a user previously consumed, such as a breakfast consisting of oatmeal topped with flaxseeds and berries. In yet another non-limiting example, a prior food preference input 180 may contain a description of a series or sequence of meals that a user previously consumed, such as all meals a user consumed for the past week, or all dinners that a user ate for a month. Information pertaining to a prior food preference input 180 may be stored within symptomatic database 132. Computing device 104 classifies using a Naïve Bayes classifier 184, a prior food preference input 180 to a food profile 144. A Naïve Bayes classifier includes any of the Naïve Bayes classifier 184 as described above in more detail in reference to FIG. 1. A Naïve Bayes classifier 184 may be trained using a second training set relating prior food preference input 180 to food profile 144. A second training set may be obtained from any of the sources as described above in more detail in reference to FIG. 1. Computing device 104 generates an ordered food element list utilizing a food profile 144.

With continued reference to FIG. 8, at step 825, computing device 104 creates a food preference menu, wherein creating the food preference menu includes grouping ordered food elements. A food preference menu 148 includes any of the food preference menu 148 as described above in more detail in reference to FIG. 1. A food preference menu 148 may contain a list of specific meals, such as suggested meals for breakfast, lunch, dinner, and/or snacks. A food preference menu 148 may contain one or more recommended options for breakfast, one or more recommended options for lunch, one or more recommended options for dinner, and/or one or more recommended options for snacks. In an embodiment, computing device 104 may group a food element 152 to generate a food preference menu 148 by pairing a first food preference with a second food preference. For instance and without limitation, computing device 104 may pair a first food preference that contains a genetically related food preference 168 containing a user's genetic preference for salty foods, with a second food preference containing a user food preference indicator that contains a user's preference for shrimp, so create a food preference menu 148 that contains a menu item containing a salt and pepper shrimp entree. Computing device 104 groups ordered food element 176 to generate a grocery ingredient list. A grocery ingredient list includes any of the grocery ingredient lists as described above in more detail in reference to FIG. 1. For example, a user may receive a transmission from computing device 104 to user device 120 containing a food preference menu 148 for a user. A user may select one or more menu items available within food preference menu 148 and transmit the selections back to computing device 104 from user device 120, utilizing any network methodology as described herein. Computing device may generate a grocery ingredient list, containing a list of ingredients that a user would need to acquire to prepare selected menu items. In an embodiment a grocery ingredient list may be modified to eliminate ingredients based on what ingredients a user may already have at home. In an embodiment, a grocery ingredient list may be displayed on remote device, so that a user could take the grocery ingredient list with them to a grocery store or place where ingredients may be sold, including any online grocery stores. Computing device 104 utilizes a food preference menu 148 to generate a nourishment strategy. A nourishment strategy includes any of the nourishment strategies as described above in more detail in reference to FIG. 1. A nourishment strategy may contain a list of suggested meals for a user, broken down by certain days or times.

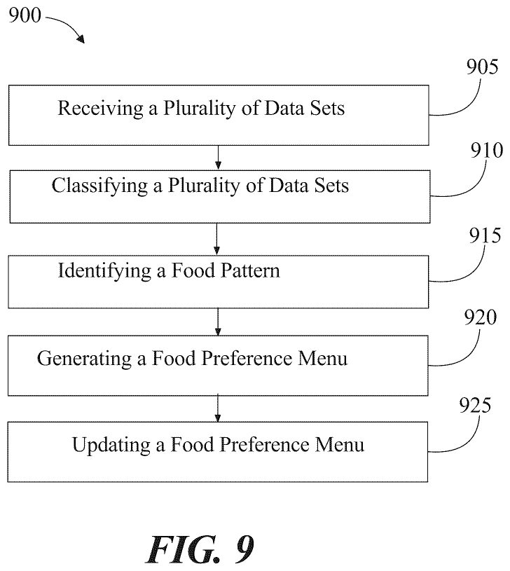

Referring now to FIG. 9, a flow diagram of an exemplary method 900 for generating food preference menu is illustrated. Method 900 includes a step 905 of receiving, using a computing device, a plurality of data sets from one or more data sources. In some embodiments, the plurality of data sets may include a prior food preference input, wherein the prior food preference input may include a food recipe. These may be implemented as disclosed with respect to FIG. 1-8.

With continued reference to FIG. 9, method 900 includes a step 910 of classifying, using the computing device, the plurality of data sets into one or more user groups, wherein classifying the plurality of data sets comprises identifying a plurality of data elements from the plurality of data sets and classifying the plurality of data sets into the one or more user groups as a function of the plurality of data elements. In some embodiments, method 900 may further include generating, using the computing device, group training data, wherein the group training data may include correlations between exemplary data sets and exemplary user groups, training, using the computing device, a group classifier using the group training data, wherein the group training data may be iteratively updated through a feedback loop and classifying, using the computing device, the plurality of data sets into the one or more user groups using the trained group classifier. In some embodiments, method 900 may further include identifying, using the computing device, at least a keyword of the plurality of data sets using a language processing module. These may be implemented as disclosed with respect to FIG. 1-8.

With continued reference to FIG. 9, method 900 includes a step 915 of identifying, using the computing device. a food pattern of the plurality of data sets in the one or more user groups. In some embodiments, the food pattern may include a genetically related food preference. In some embodiments, the food pattern may include a social conduct factor, wherein the social conduct factor may include a financial budget. In some embodiments, the food pattern may include a food preference indicator, wherein the food preference indicator may include an appetite size. In some embodiments, method 900 may further include generating, using the computing device, pattern training data, wherein the pattern training data may include correlations between exemplary data sets and exemplary food patterns, training, using the computing device, a pattern machine-learning model using the pattern training data, wherein the pattern training data may be iteratively updated through a feedback loop and determining, using the computing device, the food pattern using the trained pattern machine-learning model. These may be implemented as disclosed with respect to FIG. 1-8.

With continued reference to FIG. 9, method 900 includes a step 920 of generating, using the computing device, a food preference menu as a function of the food pattern, wherein the food preference menu comprises a nourishment strategy. In some embodiments, method 900 may further include generating, using the computing device, menu training data, wherein the menu training data may include correlations between exemplary data patterns and exemplary food preference menus, training, using the computing device, a menu machine-learning model using the menu training data, wherein the menu training data may be iteratively updated through a feedback loop and generating, using the computing device, the food preference menu using the trained menu machine-learning model. These may be implemented as disclosed with respect to FIG. 1-8.

With continued reference to FIG. 9, method 900 includes a step 925 of updating, using the computing device, the food preference menu as a function of newly collected data. In some embodiments, the newly collected entry may include feedback data related to the food preference menu. These may be implemented as disclosed with respect to FIG. 1-8.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

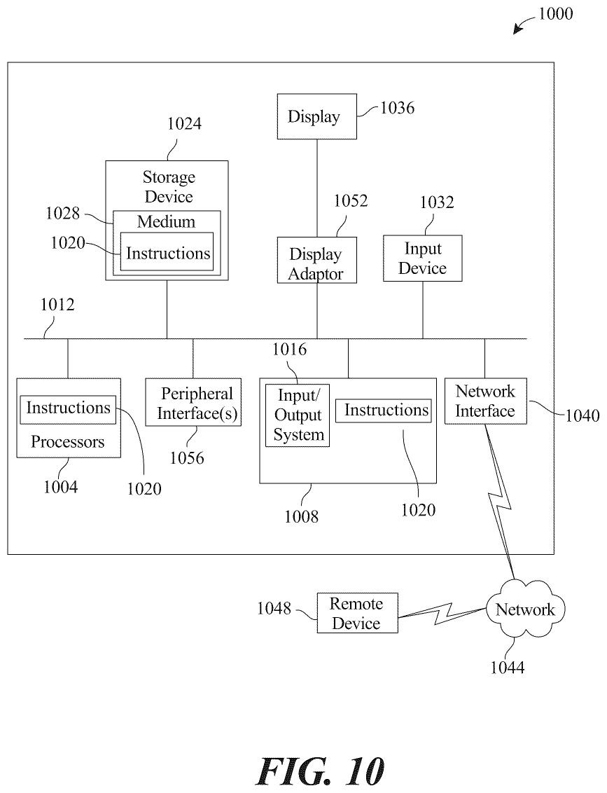

FIG. 10 shows a diagrammatic representation of one embodiment of a computing device in the exemplary form of a computer system 1000 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 1000 includes a processor 1004 and a memory 1008 that communicate with each other, and with other components, via a bus 1012. Bus 1012 may include any of several types of bus structures including, but not limited to, a memory bus, a memory controller, a peripheral bus, a local bus, and any combinations thereof, using any of a variety of bus architectures.

Memory 1008 may include various components (e.g., machine-readable media) including, but not limited to, a random access memory component, a read only component, and any combinations thereof. In one example, a basic input/output system 1016 (BIOS), including basic routines that help to transfer information between elements within computer system 1000, such as during start-up, may be stored in memory 1008. Memory 1008 may also include (e.g., stored on one or more machine-readable media) instructions (e.g., software) 1020 embodying any one or more of the aspects and/or methodologies of the present disclosure. In another example, memory 1008 may further include any number of program modules including, but not limited to, an operating system, one or more application programs, other program modules, program data, and any combinations thereof.

Computer system 1000 may also include a storage device 1024. Examples of a storage device (e.g., storage device 1024) include, but are not limited to, a hard disk drive, a magnetic disk drive, an optical disc drive in combination with an optical medium, a solid-state memory device, and any combinations thereof. Storage device 1024 may be connected to bus 1012 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 1024 (or one or more components thereof) may be removably interfaced with computer system 1000 (e.g., via an external port connector (not shown)). Particularly, storage device 1024 and an associated machine-readable medium 1028 may provide nonvolatile and/or volatile storage of machine-readable instructions, data structures, program modules, and/or other data for computer system 1000. In one example, software 1020 may reside, completely or partially, within machine-readable medium 1028. In another example, software 1020 may reside, completely or partially, within processor 1004.

Computer system 1000 may also include an input device 1032. In one example, a user of computer system 1000 may enter commands and/or other information into computer system 1000 via input device 1032. Examples of an input device 1032 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 1032 may be interfaced to bus 1012 via any of a variety of interfaces (not shown) including, but not limited to, a serial interface, a parallel interface, a game port, a USB interface, a FIREWIRE interface, a direct interface to bus 1012, and any combinations thereof. Input device 1032 may include a touch screen interface that may be a part of or separate from display 1036, discussed further below. Input device 1032 may be utilized as a user selection device for selecting one or more graphical representations in a graphical interface as described above.

A user may also input commands and/or other information to computer system 1000 via storage device 1024 (e.g., a removable disk drive, a flash drive, etc.) and/or network interface device 1040. A network interface device, such as network interface device 1040, may be utilized for connecting computer system 1000 to one or more of a variety of networks, such as network 1044, and one or more remote devices 1048 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 1044, may employ a wired and/or a wireless mode of communication. In general, any network topology may be used. Information (e.g., data, software 1020, etc.) may be communicated to and/or from computer system 1000 via network interface device 1040.

Computer system 1000 may further include a video display adapter 1052 for communicating a displayable image to a display device, such as display device 1036. Examples of a display device include, but are not limited to, a liquid crystal display (LCD), a cathode ray tube (CRT), a plasma display, a light emitting diode (LED) display, and any combinations thereof. Display adapter 1052 and display device 1036 may be utilized in combination with processor 1004 to provide graphical representations of aspects of the present disclosure. In addition to a display device, computer system 1000 may include one or more other peripheral output devices including, but not limited to, an audio speaker, a printer, and any combinations thereof. Such peripheral output devices may be connected to bus 1012 via a peripheral interface 1056. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods, systems, and software according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions, and additions may be made to that which is specifically disclosed herein without departing from the spirit and scope of the present invention.

What is claimed is:

1. A system for generating food preference menu, wherein the system comprises:
receive a plurality of data sets from one or more data sources;
classify the plurality of data sets into one or more user groups, wherein classifying the plurality of data sets comprises:
identifying a plurality of data elements from the plurality of data sets; and
classifying the plurality of data sets into the one or more user groups as a function of the plurality of data elements;
identify a food pattern of the plurality of data sets in the one or more user groups;
generate a food preference menu as a function of the food pattern, wherein the food preference menu comprises a nourishment strategy; and
update the food preference menu as a function of feedback data.

2. The system of claim 1, wherein the plurality of data sets comprises a prior food preference input, wherein the prior food preference input comprises a food recipe.

3. The system of claim 1, wherein the food pattern comprises a genetically related food preference.

4. The system of claim 1, wherein:
the food pattern comprises a social conduct factor, wherein the social conduct factor comprises a financial budget; and
generating the food preference menu as a function of the food pattern comprises generating the food preference menu as a function of the financial budget.

5. The system of claim 1, wherein:
the food pattern comprises a food preference indicator, wherein the food preference indicator comprises an appetite size; and
generating the food preference menu as a function of the food pattern comprises generating the food preference menu as a function of the appetite size.

6. The system of claim 1, wherein classifying the plurality of data sets further comprises:
generate group training data, wherein the group training data comprises correlations between exemplary data sets and exemplary user groups;
train a group classifier using the group training data, wherein the group training data is iteratively updated through a feedback loop; and
classify the plurality of data sets into the one or more user groups using the trained group classifier.

7. The system of claim 1, wherein the computing device is further configured to identify at least a keyword of the plurality of data sets using a language processing module.

8. The system of claim 1, wherein the computing device is further configured to:
generate pattern training data, wherein the pattern training data comprises correlations between exemplary data sets and exemplary food patterns;
train a pattern machine-learning model using the pattern training data, wherein the pattern training data is iteratively updated through a feedback loop; and
determine the food pattern using the trained pattern machine-learning model.

9. The system of claim 1, wherein generating a food preference menu comprises:
generate menu training data, wherein the menu training data comprises correlations between exemplary data patterns and exemplary food preference menus;
train a menu machine-learning model using the menu training data, wherein the menu training data is iteratively updated through a feedback loop; and
generate the food preference menu using the trained menu machine-learning model.

10. The system of claim 1, wherein the feedback data comprises feedback data related to the food preference menu.

11. A method for generating food preference menu, wherein the method comprises:
receiving, using a computing device, a plurality of data sets from one or more data sources;
classifying, using the computing device, the plurality of data sets into one or more user groups, wherein classifying the plurality of data sets comprises:
identifying a plurality of data elements from the plurality of data sets; and
classifying the plurality of data sets into the one or more user groups as a function of the plurality of data elements;
identifying, using the computing device, a food pattern of the plurality of data sets in the one or more user groups;
generating, using the computing device, a food preference menu as a function of the food pattern, wherein the food preference menu comprises a nourishment strategy; and
updating, using the computing device, the food preference menu as a function of feedback data.

12. The method of claim 11, wherein the plurality of data sets comprises a prior food preference input, wherein the prior food preference input comprises a food recipe.

13. The method of claim 11, wherein the food pattern comprises a genetically related food preference.

14. The method of claim 11, wherein:
the food pattern comprises a social conduct factor, wherein the social conduct factor comprises a financial budget; and
generating the food preference menu as a function of the food pattern comprises generating the food preference menu as a function of the financial budget.

15. The method of claim 11, wherein:
the food pattern comprises a food preference indicator, wherein the food preference indicator comprises an appetite size; and
generating the food preference menu as a function of the food pattern comprises generating the food preference menu as a function of the appetite size.

16. The method of claim 11, wherein classifying the plurality of data sets further comprises:
generating, using the computing device, group training data, wherein the group training data comprises correlations between exemplary data sets and exemplary user groups;
training, using the computing device, a group classifier using the group training data, wherein the group training data is iteratively updated through a feedback loop; and
classifying, using the computing device, the plurality of data sets into the one or more user groups using the trained group classifier.

17. The method of claim 11, further comprising:
identifying, using the computing device, at least a keyword of the plurality of data sets using a language processing module.

18. The method of claim 11, further comprising:
generating, using the computing device, pattern training data, wherein the pattern training data comprises correlations between exemplary data sets and exemplary food patterns;
training, using the computing device, a pattern machine-learning model using the pattern training data, wherein the pattern training data is iteratively updated through a feedback loop; and
determining, using the computing device, the food pattern using the trained pattern machine-learning model.

19. The method of claim 11, wherein generating a food preference menu comprises:
generating, using the computing device, menu training data, wherein the menu training data comprises correlations between exemplary data patterns and exemplary food preference menus;
training, using the computing device, a menu machine-learning model using the menu training data, wherein the menu training data is iteratively updated through a feedback loop; and
generating, using the computing device, the food preference menu using the trained menu machine-learning model.

20. The method of claim 11, wherein the feedback data comprises feedback data related to the food preference menu.
